(19) United States
(12) Patent Application Publication
(10) Pub. No.: US 2022/0401369 A1
(43) Pub. Date: Dec. 22, 2022

(54) SOLID DISPERSION OF PAN-RAF KINASE INHIBITOR

(71) Applicant: DAY ONE BIOPHARMACEUTICALS, INC., South San Francisco, CA (US)

(72) Inventors: Yuki KODONO, Cambridge, MA (US); Yasushi SHONO, Osaka (JP); Hirohisa TAKEUCHI, Cambridge, MA (US)

(21) Appl. No.: 17/824,237

(22) Filed: May 25, 2022

Related U.S. Application Data
(63) Continuation of application No. PCT/US20/62307, filed on Nov. 25, 2020.
(60) Provisional application No. 62/941,426, filed on Nov. 27, 2019.

(51) Int. Cl. A61K 9/20

(52) U.S. Cl. CPC A61K 9/2013; A61K 9/2027; A61K 9/2095

(57) ABSTRACT

The present disclosure provides a pharmaceutical composition comprising a solid dispersion having a mass median diameter of about 75 μm to about 400 μm, and one or more pharmaceutically acceptable excipients, wherein the solid dispersion comprises (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof, and a vinylpyrrolidone-vinyl acetate copolymer.

### CROSS REFERENCE TO RELATED APPLICATION

This application is a continuation application of PCT Patent Application No. PCT/US2020/062307, filed Nov. 25, 2020, which claims the benefit of U.S. Provisional Application No. 62/941,426 filed on Nov. 27, 2019, each of which is hereby incorporated by reference in its entirety.

### FIELD OF THE INVENTION

The present disclosure provides a pharmaceutical composition comprising a solid dispersion having mass median diameter (D₅₀) of about 75 μm to about 400 and one or more pharmaceutically acceptable excipients, wherein the solid dispersion comprises (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof, and a vinylpyrrolidone-vinyl acetate copolymer.

### BACKGROUND

The RAF kinases (A-RAF, BRAF, and C-RAF) are key components of the mitogen-activated protein kinase (MAPK) pathway that controls cell proliferation and survival signaling. See Downward J. Nature Reviews. Cancer 2003; 3(1):11-22; Wellbrock C, et al. Nature Reviews Molecular Cell Biology 2004; 5(11):875-85.

The MAP kinase (MAPK) pathway is a central signal transduction pathway that is dysregulated in a large number of developmental disorders. The MAPK pathway, which is composed of RAS, RAF, MAPK or extracellular signal-regulated kinase (MEK), and extracellular signal-regulated kinase (ERK), integrates signals from receptors on the cell surface including cancer-related receptor tyrosine kinases such as the epidermal growth factor receptor, mesenchymal-epithelial transition factor (MET), and vascular endothelial growth factor receptor (Avruch J., Biochim Biophys Acta 2007; 1773(8):1150-60). Genetic alterations in the MAPK pathway are among the most common in human cancers. Up to 60% of melanomas harbor BRAF mutations (Davies H., et al. Nature 2002; 417(6892):94954) and KRAS mutations have been estimated in roughly 60%, 30%, and 15% of pancreatic, colon, and lung tumors, respectively (Vakiani E, et al. J Pathol 2011; 223(2):219-29). BRAF mutations are also found in 40% of papillary or anaplastic thyroid cancers (Kimura E T, et al. Cancer Res 2003; 63(7):1454-7) and in a small percentage of several other types of tumor (Vakiani E, et al.). A majority of reported BRAF mutations are a substitution of glutamic acid for valine at the amino acid position of 600 (the V600E mutation). The BRAF V600E mutation constitutively activates BRAF and downstream signal transduction in the MAPK pathway (Davies H., et al.).

(R)-2-(1-(6-amino-5-chloropyrimidine-4-carboxamido)ethyl)-N-(5-chloro-4-(trifluoromethyl)pyridin-2-yl)thiazole-5-carboxamide (“Compound 1”) is a class II pan Rafkinase inhibitor useful for the treatment of Raf-mediated diseases such as cancer. WO 2009/006389 discloses Compound 1 and its use in the treatment of Raf-mediated diseases. WO 2015/148828 discloses solid dispersion extrudates comprising Compound 1 and pharmaceutical compositions thereof. WO 2013/144923 discloses methods for the treatment of non-BRAFV600E mutant melanoma in patients comprising administering Compound 1 and a MEK inhibitor.

There exists a need for improved formulations of Compound 1 for use in treating patients having cancer.

### BRIEF SUMMARY OF THE INVENTION

In one aspect, the present disclosure provides a pharmaceutical composition comprising: (1) a solid dispersion having a D₅₀ of about 75 μm to about 400 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another aspect, the present disclosure provides a pharmaceutical composition comprising: (1) a solid dispersion wherein about 70% w/w or more of the particles have a diameter of greater than or equal to about 75 μm but less than or equal to about 500 μm, i.e., the particle diameter lies between or is equal to about 75 μm and about 500 μm; and (2) one or more pharmaceutically acceptable excipients, wherein the solid dispersion comprises: (a) about 10% w/w to about 70% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof; and (b) about 30% w/w to about 90% w/w of a vinylpyrrolidone-vinyl acetate copolymer.

In another aspect, the present disclosure provides a process for preparing pharmaceutical composition comprising: (1) a solid dispersion having a D₅₀ of about 75 μm to about 400 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer, the process comprising: (1) admixing Compound 1, or a pharmaceutically acceptable salt thereof, and a vinylpyrrolidone-vinyl acetate copolymer to give a powder mixture; (2) subjecting the powder mixture to hot melt extrusion to give a solid dispersion extrudate; (3) milling the solid dispersion extrudate to give a solid dispersion having a D₅₀ of about 75 μm to about 400 μm; and (4) admixing the solid dispersion with one or more pharmaceutically acceptable excipients.

In another aspect, the present disclosure provides a solid oral dosage form, e.g., a tablet, comprising a pharmaceutical composition comprising: (1) a solid dispersion having a D₅₀ of about 75 μm to about 400 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another aspect, the present disclosure provides a method of treating cancer in a patient, comprising administering to the patient a therapeutically effective amount of a pharmaceutical composition comprising: (1) a solid dispersion having a D₅₀ of about 75 μm to about 400 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another aspect, the present disclosure provides a kit comprising a pharmaceutical composition comprising: (1) a solid dispersion having a D₅₀ of about 75 μm to about 400 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In one aspect, the present disclosure provides a pharmaceutical composition comprising: (1) a solid dispersion having a D₅₀ of about 75 μm to about 400 μm; and (2) one or more pharmaceutically acceptable excipients, wherein the solid dispersion comprises: about 10% w/w to about 70% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof; and about 30% w/w to about 90% w/w of a polymer.

In one aspect, the present disclosure provides a pharmaceutical composition comprising: (1) a solid dispersion wherein about 70% w/w or more of the particles have a diameter of greater than or equal to about 75 μm but less than or equal to about 500 μm; and (2) one or more pharmaceutically acceptable excipients, wherein the solid dispersion comprises: (a) about 10% w/w to about 70% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof; and (b) about 30% w/w to about 90% w/w of a polymer.

In one aspect, the present disclosure provides a pharmaceutical composition comprising: (1) a solid dispersion; and (2) one or more pharmaceutically acceptable excipients, wherein the solid dispersion comprises (a) Compound 1, or a pharmaceutically acceptable salt thereof; and (b) a polymer (such as a polymer suitable for hot melt extrusion). In one aspect, the present disclosure provides a pharmaceutical composition comprising a solid dispersion, wherein the solid dispersion comprises (a) Compound 1, or a pharmaceutically acceptable salt thereof; and (b) a polymer (such as a polymer suitable for hot melt extrusion). In one aspect, the present disclosure provides a solid dispersion, wherein the solid dispersion comprises (a) Compound 1, or a pharmaceutically acceptable salt thereof; and (b) a polymer (such as a polymer suitable for hot melt extrusion). In some embodiments, the polymer is a high molecular weight hydrophilic polymer. In one aspect, the present disclosure provides a pharmaceutical composition comprising: (1) a solid dispersion; and (2) one or more pharmaceutically acceptable excipients, wherein the solid dispersion comprises (a) Compound 1, or a pharmaceutically acceptable salt thereof; and (b) a high molecular weight hydrophilic polymer such as vinylpyrrolidone-vinyl acetate copolymer. In some embodiments, the solid dispersion has a D₅₀ of about 75 μm to about 400 μm. In some embodiments, about 70% w/w or more of the particles in the solid dispersion have a diameter of greater than or equal to about 75 μm but less than or equal to about 500 μm. In some embodiments, the high molecular weight hydrophilic polymer comprises at least one of polyvinylpyrrolidone (PVP, e.g., PVP-K30), vinylpyrrolidone-vinyl acetate copolymer (e.g., copovidone), cross linked polyvinyl N-pyrrolidone, polyvinyl alcohol (PVA), polysaccharide, hydroxypropyl methylcellulose (HPMC or Hypromellose; e.g., HPMC-E5), hydroxyethyl cellulose (HEC), hydroxypropyl cellulose (HPC), polyethylene oxide, hydroxypropyl-β-cyclodextrin (HP-β-CD), sulfobutylether-β-cyclodextrin, hydroxypropyl methylcellulose acetate succinate (HPMC-AS-HF), polyethylene glycol (PEG), polyvinyl caprolactam-polyvinyl acetate-polyethylene glycol graft copolymer (PVAc-PVCap-PEG, e.g., sold under the trade name Soluplus), polysaccharide, or a combination thereof. In some embodiments, the high molecular weight hydrophilic polymer is PVP, copovidone, crospovidone, HPMC, or a combination thereof. In some embodiments, the high molecular weight hydrophilic polymer comprises HPMC and crospovidone. In some embodiments, the high molecular weight hydrophilic polymer comprises HPMC and PVP. In some embodiments, the high molecular weight hydrophilic polymer comprises HPMC and copovidone. In some embodiments, the high molecular weight hydrophilic polymer comprises copovidone and crospovidone.

In one aspect, the present disclosure provides a method of treating cancer in a patient, comprising administering to the patient a therapeutically effective amount of a pharmaceutical composition comprising: (1) a solid dispersion; and (2) one or more pharmaceutically acceptable excipients, wherein the solid dispersion comprises (a) Compound 1, or a pharmaceutically acceptable salt thereof; and (b) a polymer.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1 is four line graphs showing the dissolution profile of hot melt extrusion (HME) loaded tablets (USP Apparatus 2, Paddle 75 rpm, 900 mL, 0.3-0.45% CTAB in pH 1.1).

FIG. 2 is two line graphs showing the tablet properties of HME loaded tablets.

FIG. 3 is three line graphs showing the dissolution profile of tablet formulations (USP Apparatus 2, Paddle 75 rpm, 900 mL, 0.35 CTAB in pH 1.1).

FIG. 4 is two line graphs showing the tablet properties of tablet formulations.

FIG. 5 is a line graph showing the dissolution profile of a prototype 150 mg HME (50%) tablet (USP Apparatus 2, Paddle 75 rpm, 900 mL, 0.3% CTAB in pH 1.1). “T2” refers to HME (40%) tablet.

FIG. 6 is three lines graphs and three illustrations showing the dissolution profile Compound 1 tablets (100 mg) made with different particle sizes of HME (40% HME in tablet).

FIG. 7 is four line graphs showing the dissolution profile of a prototype HME (50%) (referred as the “T3”) core tablet (20, 70, 100 and 150 mg) (USP Apparatus 2, Paddle 75 rpm, 900 mL, 0.3% CTAB in pH 1.1). “T2” refers to HME (40%) core tablet.

FIG. 8 is two line graphs showing the dissolution profiles of scale-up HME (50%) core tablets (100 and 150 mg).

FIG. 9A is fourteen line graphs showing the dissolution profile of certain tablet formulations of Table 14. (USP Apparatus 2, Paddle 75 rpm, 900 mL, 0.35% CTAB in pH 1.1).

FIG. 9B is eight line graphs showing the dissolution profile of certain tablet formulations of Table 14. (USP Apparatus 2, Paddle 75 rpm, 900 mL, 0.35% CTAB in pH 1.1).

### DETAILED DESCRIPTION OF THE INVENTION

In one aspect, the present disclosure provides a pharmaceutical composition comprising: (1) a solid dispersion and (2) one or more pharmaceutically acceptable excipients, wherein the solid dispersion comprises Compound 1, or a pharmaceutically acceptable salt thereof; and (b) a polymer such as a high molecular weight hydrophilic polymer. In one aspect, described herein is a solid dispersion that comprises Compound 1, or a pharmaceutically acceptable salt thereof, and a polymer such as a high molecular weight hydrophilic polymer. In some embodiments, the polymer is a polymer used in hot melt extrusion. In some embodiments, the solid dispersion has a D₅₀, D₉₀, and/or D₁₀ value as described herein. In some embodiments, the solid dispersion comprises particles that have a D₅₀, D₉₀, and/or D₁₀ value as described herein. In some embodiments, the solid dispersion comprises particles, and at least 30%, 40%, 50%, 60%, 70%, 80%, 90%, 95%, 99%, or 100% of the particles have a D₅₀, D₉₀, and/or D₁₀ value as described herein.

In one embodiment, the present disclosure provides a pharmaceutical composition comprising: (1) a solid dispersion having a D₅₀ of about 75 μm to about 400 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion wherein about 70% w/w or more of the particles have a diameter of greater than or equal to about 75 μm; and (2) one or more pharmaceutically acceptable excipients, wherein the solid dispersion comprises: (a) about 10% w/w to about 70% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion wherein about 70% w/w or more of the particles have a diameter of greater than or equal to about 75 μm but less than or equal to about 500 μm, i.e., the particle diameter lies between or is equal to about 75 μm and about 500 μm; and (2) one or more pharmaceutically acceptable excipients, wherein the solid dispersion comprises: (a) about 10% w/w to about 70% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion having a D₁₀ of about 10 μm to about 200 μm; and (2) one or more pharmaceutically acceptable excipients, wherein the solid dispersion comprises: (a) about 10 w/w to about 70% w/w of Compound 1, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion having a D₉₀ of about 100 μm to about 1000 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion having: (i) a D₁₀ of about 10 μm to about 200 μm; and (ii) a D₉₀ of about 100 μm to about 1000 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion having: (i) a D₁₀ of about 10 μm to about 200 μm; and (ii) a D₅₀ of about 75 μm to about 400 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion having: (i) a D₅₀ of about 75 μm to about 400 μm; and (ii) a D₉₀ of about 100 μm to about 1000 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another embodiment, the present disclosure provides a pharmaceutical composition comprising: (1) a solid dispersion having: (i) a D₁₀ of about 10 μm to about 200 μm; (ii) a D₅₀ of about 75 μm to about 400 μm; and (iii) a D₉₀ of about 100 μm to about 1000 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In one aspect, the present disclosure provides a pharmaceutical composition comprising a solid dispersion, wherein the solid dispersion comprises (a) Compound 1, or a pharmaceutically acceptable salt thereof; and (b) a polymer (such as a polymer suitable for hot melt extrusion).

Collectively, the pharmaceutical compositions described above are referred to herein as a “Composition of the Disclosure.”

In another embodiment, the present disclosure provides a Composition of the Disclosure, wherein the solid dispersion has a D₅₀ of about 85 μm to about 250 μm. In another embodiment, the solid dispersion has a D₅₀ of about 95 μm to about 150 μm. In another embodiment, the solid dispersion has a D₅₀ of about 75 μm to about 150 μm. In another embodiment, the solid dispersion has a D₅₀ of about 75 μm to about 250 μm. In another embodiment, the solid dispersion has a D₅₀ of about 75 μm to about 400 μm. In another embodiment, the solid dispersion has a D₅₀ of about 75 μm to about 500 μm. In another embodiment, the solid dispersion has a D₅₀ of about 75 μm to about 600 μm. In another embodiment, the solid dispersion has a D₅₀ of about 150 μm to about 250 μm. In another embodiment, the solid dispersion has a D₅₀ of about 150 μm to about 400 μm. In another embodiment, the solid dispersion has a D₅₀ of about 150 μm to about 600 μm. In another embodiment, the solid dispersion has a D₅₀ of about 100 μm to about 110 μm. In another embodiment, the solid dispersion has a D₅₀ of about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 220 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, or about 400 μm. In another embodiment, the solid dispersion has a D₅₀ of at least about 50 μm, at least about 75 μm, at least about 80 μm, at least about 85 μm, at least about 90 μm, at least about 95 μm, at least about 100 μm, at least about 110 μm, at least about 120 μm, at least about 125 μm, at least about 150 μm, at least about 175 μm, at least about 200 μm, at least about 250 μm, at least about 300 μm, at least about 350 μm, or at least about 400 μm. In another embodiment, the solid dispersion has a D₅₀ of at most about 75 μm, at most about 100 μm, at most about 125 μm, at most about 150 μm, at most about 200 μm, at most about 250 μm, at most about 300 μm, at most about 350 μm, at most about 400 μm, at most about 500 μm, or at most about 800 μm. In another embodiment, the solid dispersion has a D₅₀ of about 105 μm. In one embodiment, the D₅₀ is determined by sieving particle size analysis.

In another embodiment, the present disclosure provides a Composition of the Disclosure, wherein the solid dispersion has a D₁₀ of about 25 μm to about 150 μm. In another embodiment, the solid dispersion has a D₁₀ of about 25 μm to about 100 μm. In another embodiment, the solid dispersion has a D₁₀ of about 25 μm to about 75 μm. In another embodiment, the solid dispersion has a D₁₀ of about 25 μm to about 50 μm. In another embodiment, the solid dispersion has a D₁₀ of about 25 μm to about 50 μm. In another embodiment, the solid dispersion has a D₁₀ of about 10 μm, about 20 μm, about 25 μm, about 30 μm, about 35 μm, about 40 μm, about 45 μm, about 50 μm, about 60 μm, about 70 μm, about 75 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm. In another embodiment, the solid dispersion has a D₁₀ of about 30 μm.

In another embodiment, the present disclosure provides a Composition of the Disclosure, wherein the solid dispersion has a D₉₀ of about 100 μm to about 500 μm. In another embodiment, the solid dispersion has a D₉₀ of about 100 μm to about 250 μm. In another embodiment, the solid dispersion has a D₉₀ of about 100 μm to about 200 μm. In another embodiment, the solid dispersion has a D₉₀ of about 100 μm to about 400 μm. In another embodiment, the solid dispersion has a D₉₀ of about 150 μm to about 400 μm. In another embodiment, the solid dispersion has a D₉₀ of about 250 μm to about 400 μm. In another embodiment, the solid dispersion has a D₉₀ of about 100 μm to about 600 μm. In another embodiment, the solid dispersion has a D₉₀ of about 250 μm to about 800 μm. In another embodiment, the solid dispersion has a D₉₀ of about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm about 850 μm, about 875 μm, about 900 μm, about 925 μm, about 950 μm, about 975 μm, or about 1000 μm. In another embodiment, the solid dispersion has a D₉₀ of about 150 μm.

In another embodiment, the present disclosure provides a Composition of the Disclosure comprising: (1) a solid dispersion having: (i) a D₁₀ of about 30 μm; (ii) a D₅₀ of about 105 μm; and (iii) a D₉₀ of about 150 μm; and (2) one or more pharmaceutically acceptable excipients, wherein the solid dispersion comprises: (a) about 10% w/w to about 70% w/w of Compound 1, or a pharmaceutically acceptable salt thereof; and (b) about 30% w/w to about 90% w/w of a vinylpyrrolidone-vinyl acetate copolymer.

In another embodiment, the present disclosure provides a Composition of the Disclosure, wherein about 75% w/w or more of the particles have a diameter that is greater than or equal to about 75 μm. In another embodiment, the present disclosure provides a Composition of the Disclosure, wherein about 80% w/w or more of the particles have a diameter that is greater than or equal to about 75 μm. In another embodiment, the present disclosure provides a Composition of the Disclosure, wherein about 85% w/w or more of the particles have a diameter that is greater than or equal to about 75 μm. In another embodiment, the present disclosure provides a Composition of the Disclosure, wherein about 90% w/w or more of the particles have a diameter that is greater than or equal to about 75 μm. In another embodiment, the present disclosure provides a Composition of the Disclosure, wherein about 95% w/w or more of the particles have a diameter that is greater than or equal to about 75 μm.

In another embodiment, the present disclosure provides a Composition of the Disclosure, wherein about 70% w/w or more of the particles have a diameter that lies between or is equal to about 75 μm and about 250 μm. In another embodiment, the particle diameter lies between or is equal to about 75 μm and about 150 μm.

In another embodiment, the present disclosure provides a Composition of the Disclosure, wherein about 75 w/w or more of the particles have a diameter that lies between or is equal to about 75 μm and about 500 μm. In another embodiment, the particle diameter lies between or is equal to about 75 μm and about 250 μm. In another embodiment, the particle diameter lies between or is equal to about 75 μm and about 150 μm.

In another embodiment, the present disclosure provides a Composition of the Disclosure, wherein about 80% w/w or more of the particles have a diameter that lies between or is equal to about 75 μm and about 500 μm. In another embodiment, the particle diameter lies between or is equal to about 75 μm and about 250 μm. In another embodiment, the particle diameter lies between or is equal to about 75 μm and about 150 μm.

In another embodiment, the present disclosure provides a Composition of the Disclosure, wherein about 85 w/w or more of the particles have a diameter that lies between or is equal to about 75 μm and about 500 μm. In another embodiment, the particle diameter lies between or is equal to about 75 μm and about 250 μm. In another embodiment, the particle diameter lies between or is equal to about 75 μm and about 150 μm.

In another embodiment, the present disclosure provides a Composition of the Disclosure, wherein about 90% w/w or more of the particles have a diameter that lies between or is equal to about 75 μm and about 500 μm. In another embodiment, the particle diameter lies between or is equal to about 75 μm and about 250 μm. In another embodiment, the particle diameter lies between or is equal to about 75 μm and about 150 μm.

In another embodiment, the present disclosure provides a Composition of the Disclosure, wherein about 95% w/w or more of the particles have a diameter that lies between or is equal to about 75 μm and about 500 μm. In another embodiment, the particle diameter lies between or is equal to about 75 μm and about 250 μm. In another embodiment, the particle diameter lies between or is equal to about 75 μm and about 150 μm.

In another embodiment, the present disclosure provides a Composition of the Disclosure, wherein the solid dispersion comprises about 10% w/w to about 90% w/w of Compound 1, or a pharmaceutically acceptable salt thereof. In another embodiment, the solid dispersion comprises about 20% w/w to about 80% w/w of Compound 1, or a pharmaceutically acceptable salt thereof. In another embodiment, the solid dispersion comprises about 30% w/w to about 75% w/w of Compound 1, or a pharmaceutically acceptable salt thereof. In another embodiment, the present disclosure provides a Composition of the Disclosure, wherein the solid dispersion comprises about 35% w/w to about 65% w/w of Compound 1, or a pharmaceutically acceptable salt thereof. In another embodiment, the present disclosure provides a Composition of the Disclosure, wherein the solid dispersion comprises about 35% w/w to about 75 w/w of Compound 1, or a pharmaceutically acceptable salt thereof. In another embodiment, the solid dispersion comprises about 40% w/w to about 60% w/w of Compound 1, or a pharmaceutically acceptable salt thereof. In another embodiment, the solid dispersion comprises about 40% w/w to about 70% w/w of Compound 1, or a pharmaceutically acceptable salt thereof. In another embodiment, the solid dispersion comprises about 45% w/w to about 55 w/w of Compound 1, or a pharmaceutically acceptable salt thereof. In another embodiment, the solid dispersion comprises about 45% w/w to about 65% w/w of Compound 1, or a pharmaceutically acceptable salt thereof. In another embodiment, the solid dispersion comprises about 50% w/w to about 60% w/w of Compound 1, or a pharmaceutically acceptable salt thereof. In another embodiment, the solid dispersion comprises about 55% w/w to about 65% w/w of Compound 1, or a pharmaceutically acceptable salt thereof. In another embodiment, the solid dispersion comprises about 50% w/w to about 65% w/w of Compound 1, or a pharmaceutically acceptable salt thereof. In another embodiment, the solid dispersion comprises about 55% w/w to about 60% w/w of Compound 1, or a pharmaceutically acceptable salt thereof. In another embodiment, the solid dispersion comprises about 50 w/w of Compound 1, or a pharmaceutically acceptable salt thereof.

In another embodiment, the present disclosure provides a Composition of the Disclosure, wherein the solid dispersion comprises about 10% w/w to about 90% w/w of a polymer (e.g., a high molecular weight hydrophilic polymer). In another embodiment, the present disclosure provides a Composition of the Disclosure, wherein the solid dispersion comprises about 20% w/w to about 80% w/w of a polymer (e.g., a high molecular weight hydrophilic polymer). In another embodiment, the present disclosure provides a Composition of the Disclosure, wherein the solid dispersion comprises about 30% w/w to about 75 w/w of a polymer (e.g., a high molecular weight hydrophilic polymer). In another embodiment, the present disclosure provides a Composition of the Disclosure, wherein the solid dispersion comprises about 35% w/w to about w/w of a polymer (e.g., a high molecular weight hydrophilic polymer). In another embodiment, the present disclosure provides a Composition of the Disclosure, wherein the solid dispersion comprises about 25% w/w to about 50 w/w of a polymer. In another embodiment, the solid dispersion comprises about 40% w/w to about 70% w/w of a polymer (e.g., a high molecular weight hydrophilic polymer). In another embodiment, the solid dispersion comprises about 45% w/w to about 65% w/w of a polymer (e.g., a high molecular weight hydrophilic polymer). In another embodiment, the present disclosure provides a Composition of the Disclosure, wherein the solid dispersion comprises about 50% w/w to about 65% w/w of a polymer (e.g., a high molecular weight hydrophilic polymer). In another embodiment, the present disclosure provides a Composition of the Disclosure, wherein the solid dispersion comprises about 50% w/w to about 80% w/w of a polymer. In another embodiment, the solid dispersion comprises about 50% w/w of a polymer (e.g., a high molecular weight hydrophilic polymer). In some embodiments, the polymer is a polymer in Table 1 or the like. In some embodiments, the polymer is a polymer used in hot melt extrusion. In another embodiment, the present disclosure provides a Composition of the Disclosure, wherein the solid dispersion comprises a polymer used in hot melt extrusion (HME). Exemplary commonly used polymers and co-polymers for HME include polyvinylpyrrolidone (PVP), polyvinylpyrrolidone-vinyl acetate (PVP-VA), poly (ethylene-co-vinyl acetate), polyethylene glycol (PEG), cellulose-esters, cellulose-acrylates, polyethylene oxides (PEOs), poly-methacrylate derivatives, poloxamers, hydroxypropylcellulose (HPC), polylactic acid (PLA), poly(glycolide) (PGA), and poly(lactide-co-glycolide) (PLGA).

In another embodiment, the present disclosure provides a Composition of the Disclosure, wherein the solid dispersion comprises about 10% w/w to about 90% w/w of a vinylpyrrolidone-vinyl acetate copolymer. In another embodiment, the present disclosure provides a Composition of the Disclosure, wherein the solid dispersion comprises about 20% w/w to about 80% w/w of a vinylpyrrolidone-vinyl acetate copolymer. In another embodiment, the present disclosure provides a Composition of the Disclosure, wherein the solid dispersion comprises about 30% w/w to about 75% w/w of a vinylpyrrolidone-vinyl acetate copolymer. In another embodiment, the present disclosure provides a Composition of the Disclosure, wherein the solid dispersion comprises about 35% w/w to about 65% w/w of a vinylpyrrolidone-vinyl acetate copolymer. In another embodiment, the present disclosure provides a Composition of the Disclosure, wherein the solid dispersion comprises about 35% w/w to about 75 w/w of a vinylpyrrolidone-vinyl acetate copolymer. In another embodiment, the solid dispersion comprises about 40% w/w to about 60% w/w of a vinylpyrrolidone-vinyl acetate copolymer. In another embodiment, the solid dispersion comprises about 40% w/w to about 70% w/w of a vinylpyrrolidone-vinyl acetate copolymer. In another embodiment, the solid dispersion comprises about 45% w/w to about 55% w/w of a vinylpyrrolidone-vinyl acetate copolymer. In another embodiment, the solid dispersion comprises about 45% w/w to about 65 w/w of a vinylpyrrolidone-vinyl acetate copolymer. In another embodiment, the present disclosure provides a Composition of the Disclosure, wherein the solid dispersion comprises about 50 w/w to about 65% w/w of a vinylpyrrolidone-vinyl acetate copolymer. In another embodiment, the solid dispersion comprises about 50% w/w of a vinylpyrrolidone-vinyl acetate copolymer.

In another embodiment, the present disclosure provides a Composition of the Disclosure, wherein the solid dispersion comprises about 10% w/w to about 90% w/w of crospovidone. In another embodiment, the present disclosure provides a Composition of the Disclosure, wherein the solid dispersion comprises about 20% w/w to about 80% w/w of crospovidone. In another embodiment, the present disclosure provides a Composition of the Disclosure, wherein the solid dispersion comprises about 30% w/w to about 75% w/w of crospovidone. In another embodiment, the present disclosure provides a Composition of the Disclosure, wherein the solid dispersion comprises about 35 w/w to about 75% w/w of crospovidone. In another embodiment, the solid dispersion comprises about 40% w/w to about 70% w/w of crospovidone. In another embodiment, the solid dispersion comprises about 45% w/w to about 65% w/w of crospovidone. In another embodiment, the present disclosure provides a Composition of the Disclosure, wherein the solid dispersion comprises about 50% w/w to about 65 w/w of crospovidone. In another embodiment, the solid dispersion comprises about 50% w/w of crospovidone.

In another embodiment, the present disclosure provides a Composition of the Disclosure, wherein the solid dispersion comprises about 10% w/w to about 90% w/w of HPMCAS-LG. In another embodiment, the present disclosure provides a Composition of the Disclosure, wherein the solid dispersion comprises about 20% w/w to about 80% w/w of HPMCAS-LG. In another embodiment, the present disclosure provides a Composition of the Disclosure, wherein the solid dispersion comprises about 30% w/w to about 75% w/w of HPMCAS-LG. In another embodiment, the present disclosure provides a Composition of the Disclosure, wherein the solid dispersion comprises about 35% w/w to about 75% w/w of HPMCAS-LG. In another embodiment, the solid dispersion comprises about 40% w/w to about 70% w/w of HPMCAS-LG. In another embodiment, the solid dispersion comprises about 45% w/w to about 65% w/w of HPMCAS-LG. In another embodiment, the present disclosure provides a Composition of the Disclosure, wherein the solid dispersion comprises about 50% w/w to about 65% w/w of HPMCAS-LG. In another embodiment, the solid dispersion comprises about 50% w/w of HPMCAS-LG.

In another embodiment, the present disclosure provides a Composition of the Disclosure, wherein the solid dispersion comprises about 10% w/w to about 90% w/w of HPMCAS-MG. In another embodiment, the present disclosure provides a Composition of the Disclosure, wherein the solid dispersion comprises about 20% w/w to about 80% w/w of HPMCAS-MG. In another embodiment, the present disclosure provides a Composition of the Disclosure, wherein the solid dispersion comprises about 30% w/w to about 75% w/w of HPMCAS-MG. In another embodiment, the present disclosure provides a Composition of the Disclosure, wherein the solid dispersion comprises about 35% w/w to about 75 w/w of HPMCAS-MG. In another embodiment, the solid dispersion comprises about 40% w/w to about 70% w/w of HPMCAS-MG. In another embodiment, the solid dispersion comprises about 45% w/w to about 65% w/w of HPMCAS-MG. In another embodiment, the present disclosure provides a Composition of the Disclosure, wherein the solid dispersion comprises about 50% w/w to about 65% w/w of HPMCAS-MG. In another embodiment, the solid dispersion comprises about 50% w/w of HPMCAS-MG.

In another embodiment, the present disclosure provides a Composition of the Disclosure, wherein the solid dispersion comprises about 10% w/w to about 90% w/w of HPMCAS-HG. In another embodiment, the present disclosure provides a Composition of the Disclosure, wherein the solid dispersion comprises about 20% w/w to about 80% w/w of HPMCAS-HG. In another embodiment, the present disclosure provides a Composition of the Disclosure, wherein the solid dispersion comprises about 30% w/w to about 75% w/w of HPMCAS-HG. In another embodiment, the present disclosure provides a Composition of the Disclosure, wherein the solid dispersion comprises about 35% w/w to about 75% w/w of HPMCAS-HG. In another embodiment, the solid dispersion comprises about 40% w/w to about 70% w/w of HPMCAS-HG. In another embodiment, the solid dispersion comprises about 45% w/w to about 65% w/w of HPMCAS-HG. In another embodiment, the present disclosure provides a Composition of the Disclosure, wherein the solid dispersion comprises about 50% w/w to about 65% w/w of HPMCAS-HG. In another embodiment, the solid dispersion comprises about 50% w/w of HPMCAS-HG.

In another embodiment, the present disclosure provides a Composition of the Disclosure, wherein the solid dispersion comprises about 10% w/w to about 90% w/w of polyvinyl caprolactam-polyvinyl acetate-polyethylene glycol graft copolymer, which is sold under the trade name of Soluplus®. In another embodiment, the present disclosure provides a Composition of the Disclosure, wherein the solid dispersion comprises about 20% w/w to about 80% w/w of polyvinyl caprolactam-polyvinyl acetate-polyethylene glycol graft copolymer. In another embodiment, the present disclosure provides a Composition of the Disclosure, wherein the solid dispersion comprises about 30% w/w to about 75% w/w of polyvinyl caprolactam-polyvinyl acetate-polyethylene glycol graft copolymer. In another embodiment, the present disclosure provides a Composition of the Disclosure, wherein the solid dispersion comprises about 35% w/w to about 75 w/w of polyvinyl caprolactam-polyvinyl acetate-polyethylene glycol graft copolymer. In another embodiment, the solid dispersion comprises about 40% w/w to about 70% w/w of polyvinyl caprolactam-polyvinyl acetate-polyethylene glycol graft copolymer. In another embodiment, the solid dispersion comprises about 45% w/w to about 65% w/w of polyvinyl caprolactam-polyvinyl acetate-polyethylene glycol graft copolymer. In another embodiment, the present disclosure provides a Composition of the Disclosure, wherein the solid dispersion comprises about 50 w/w to about 65% w/w of polyvinyl caprolactam-polyvinyl acetate-polyethylene glycol graft copolymer. In another embodiment, the solid dispersion comprises about w/w of polyvinyl caprolactam-polyvinyl acetate-polyethylene glycol graft copolymer.

In another embodiment the present disclosure provides a Composition of the Disclosure, wherein the solid dispersion comprises about 45% w/w to about 55 w/w of Compound 1 and about 45 w/w to about 55 w/w of a vinylpyrrolidone-vinyl acetate copolymer.

In another embodiment the present disclosure provides a Composition of the Disclosure, wherein the solid dispersion consists of about 40% w/w of Compound 1 and about 60% w/w of a vinylpyrrolidone-vinyl acetate copolymer.

In another embodiment the present disclosure provides a Composition of the Disclosure, wherein the solid dispersion consists of about 45% w/w of Compound 1 and about 55% w/w of a vinylpyrrolidone-vinyl acetate copolymer.

In another embodiment the present disclosure provides a Composition of the Disclosure, wherein the solid dispersion consists of about 50% w/w of Compound 1 and about 50% w/w of a vinylpyrrolidone-vinyl acetate copolymer.

In another embodiment the present disclosure provides a Composition of the Disclosure, wherein the solid dispersion consists of about 55% w/w of Compound 1 and about 45 w/w of a vinylpyrrolidone-vinyl acetate copolymer.

In another embodiment the present disclosure provides a Composition of the Disclosure, wherein the solid dispersion consists of about 60% w/w of Compound 1 and about 40% w/w of a vinylpyrrolidone-vinyl acetate copolymer.

In another embodiment, the present disclosure provides a Composition of the Disclosure, wherein the vinylpyrrolidone-vinyl acetate copolymer is copovidone. In another embodiment, the vinylpyrrolidone-vinyl acetate copolymer is Kollidon® VA 64.

In one aspect, the present disclosure provides a pharmaceutical composition comprising a solid dispersion, wherein the solid dispersion comprises (a) Compound 1, or a pharmaceutically acceptable salt thereof; and (b) a polymer described herein.

A pharmaceutical composition described herein can comprise about 5% w/w to about 100% w/w of a described solid dispersion. In some embodiments, the composition comprises about 5% w/w to about 95% w/w of the solid dispersion. In some embodiments, the composition comprises about 20% w/w to about 80% w/w of the solid dispersion. In some embodiments, the composition comprises about 30% w/w to about 70% w/w of the solid dispersion. In some embodiments, the composition comprises about 40% w/w to about 60% w/w of the solid dispersion. In some embodiments, the composition comprises about 45% w/w to about 55% w/w of the solid dispersion. In some embodiments, the composition comprises about 30% w/w to about 50% w/w of the solid dispersion. In some embodiments, the composition comprises about 50% w/w to about 70% w/w of the solid dispersion. In some embodiments, the composition comprises about 30% w/w to about 60% w/w of the solid dispersion. In some embodiments, the composition comprises about 40% w/w of the solid dispersion. In some embodiments, the composition comprises about 50% w/w of the solid dispersion. In some embodiments, the composition comprises about 60% w/w of the solid dispersion. In some embodiments, the composition comprises at least about 20% w/w, at least about 30% w/w, at least about 35% w/w, at least about 40% w/w, at least about 45% w/w, at least about 50% w/w, at least about 55% w/w, at least about 60% w/w, or at least about 70% w/w of the solid dispersion. In some embodiments, the composition comprises at most about 30% w/w, at most about 35% w/w, at most about 40% w/w, at most about 45% w/w, at most about 50% w/w, at most about 55% w/w, at most about 60% w/w, at most about 70% w/w, at most about 80% w/w, at most about 90% w/w, or at most about 99% w/w of the solid dispersion.

In another embodiment, the present disclosure provides a Composition of the Disclosure, wherein the one or more pharmaceutically acceptable excipients comprise a filler, a disintegrant, a glidant, and/or a lubricant.

In another embodiment, the present disclosure provides a Composition of the Disclosure, wherein the one or more pharmaceutically acceptable excipients comprise a filler. In another embodiment, the filler is microcrystalline cellulose (MCC), e.g., MCC PH101, MCC UF702, MCC UF711, MCC OF. In another embodiment, the microcrystalline cellulose is MCC UF711. In some embodiments, the filler is dibasic calcium phosphate anhydrous. In some embodiments, the filler is sodium dodecyl sulfate. In some embodiments, the filler is sugar (e.g., glucose, sucrose, mannitol). In some embodiments, the filler is calcium carbonate. The filler can be present in the composition at about 10% w/w to about 90% w/w. The filler can be present in the composition at about 30% w/w to about 80% w/w. The filler can be present in the composition at about 40% w/w to about 70% w/w. The filler can be present in the composition at about 50% w/w to about 60% w/w.

In another embodiment, the present disclosure provides a Composition of the Disclosure, wherein the one or more pharmaceutically acceptable excipients comprise a disintegrant. In some embodiments, the disintegrant is a starch (e.g., maize starch, wheat starch, potato starch, mannitol-starch). In some embodiments, the disintegrant is croscarmellose sodium. In some embodiments, the disintegrant is sodium carboxyl methyl cellulose. In some embodiments, the disintegrant is sodium starch glycolate. In some embodiments, the disintegrant is lactose crystals (e.g., milled lactose, coarse lactose). In some embodiments, the disintegrant is α-lactose monohydrate. In some embodiments, the disintegrant is a polysaccharide (e.g., soy polysaccharide). The disintegrant can be present in the composition at about 1% w/w to about 20% w/w. The disintegrant can be present in the composition at about 5% w/w to about 15% w/w. The disintegrant can be present in the composition at about 5 w/w to about 10% w/w. The disintegrant can be present in the composition at about 8% w/w.

In another embodiment, the present disclosure provides a Composition of the Disclosure, wherein the one or more pharmaceutically acceptable excipients comprise a glidant. In another embodiment, the glidant is colloidal silicon dioxide (e.g., fumed silica, silica derivatives, Syloid®). In another embodiment, the glidant is cornstarch. In another embodiment, the glidant is a talc. In another embodiment, the glidant is hydrated sodium sulfoaluminate. The glidant can be present in the composition at about 0.1% w/w to about 5% w/w. The glidant can be present in the composition at about 0.2% w/w to about 3% w/w. The glidant can be present in the composition at about 0.5% w/w.

In another embodiment, the present disclosure provides a Composition of the Disclosure, wherein the one or more pharmaceutically acceptable excipients comprise a lubricant. In some embodiments, the lubricant is magnesium stearate. In some embodiments, the lubricant is stearate acid. In some embodiments, the lubricant is sodium stearyl fumerate. In some embodiments, the lubricant is a vegetable stearate. In some embodiments, the lubricant is stearate acid. In some embodiments, the lubricant is a glyceryl/polyethylene glycol dibehenate. In some embodiments, the lubricant is a hydrogenated vegetable oil (e.g., cottonseed oil). The lubricant can be present in the composition at about 0.1% w/w to about 5 w/w. The lubricant can be present in the composition at about 0.2% w/w to about 3% w/w. The lubricant can be present in the composition at about 0.5% w/w.

In another embodiment, the present disclosure provides a Composition of the Disclosure comprising about 40% w/w to about 90% w/w of one or more pharmaceutically acceptable excipients. In another embodiment, the Composition of the Disclosure comprises about 50 w/w to about 80% w/w of one or more pharmaceutically acceptable excipients. In another embodiment, the Composition of the Disclosure comprises about 50% w/w to about 70% w/w of one or more pharmaceutically acceptable excipients. In another embodiment, the Composition of the Disclosure comprises about 30% w/w to about 50% w/w of one or more pharmaceutically acceptable excipients. In another embodiment, the Composition of the Disclosure comprises about 50% w/w of one or more pharmaceutically acceptable excipients.

In another embodiment, the present disclosure provides a Composition of the Disclosure, wherein Compound 1 is amorphous.

In another embodiment, the present disclosure provides a process for preparing a Composition of the Disclosure, the process comprising: (1) admixing Compound 1, or a pharmaceutically acceptable salt thereof, and a vinylpyrrolidone-vinyl acetate copolymer to give a powder mixture; (2) subjecting the powder mixture to hot melt extrusion to give a solid dispersion extrudate; (3) milling the solid dispersion extrudate to give a solid dispersion having the desired D₅₀, e.g., a D₅₀ of about 75 μm to about 400 μm; and (4) admixing the solid dispersion with one or more pharmaceutically acceptable excipients. In another embodiment, the solid dispersion extrudate is milled to give a solid dispersion having D₅₀ of about 85 μm to about 250 μm. In another embodiment, the solid dispersion extrudate is milled to give a solid dispersion having D₅₀ of about 95 μm to about 150 μm. In another embodiment, the solid dispersion extrudate is milled to give a solid dispersion having D₅₀ of about 105 μm.

In another embodiment, the present disclosure provides a solid oral dosage form, e.g., a tablet, comprising a Composition of the Disclosure. In another embodiment, solid oral dosage form comprises about 1 mg to about 300 mg of Compound 1. In another embodiment, solid oral dosage form comprises about 5 mg to about 250 mg of Compound 1. In another embodiment, solid oral dosage form comprises about 20 mg to about 100 mg of Compound 1. In another embodiment, solid oral dosage form comprises about 50 mg to about 150 mg of Compound 1. In another embodiment, solid oral dosage form comprises about 150 mg to about 250 mg of Compound 1. In another embodiment, solid oral dosage form comprises about 20 mg to about 20 mg of Compound 1. In another embodiment, the solid oral dosage form comprises at least about 20 mg, at least about 25 mg, at least about 50 mg, at least about 75 mg, at least about 100 mg, at least about 125 mg, at least about 150 mg, at least about 200 mg, at least about 250 mg, at least about 300 mg, or at least about 400 mg of Compound 1. In another embodiment, the solid oral dosage form comprises at most about 50 mg, at most about 75 mg, at most about 100 mg, at most about 125 mg, at most about 150 mg, at most about 200 mg, at most about 250 mg, at most about 300 mg, at most about 400 mg, at most about 500 mg, or at most about 600 mg of Compound 1. In another embodiment, the solid oral dosage form comprises about 20 mg, about 25 mg, about 50 mg, about 75 mg, about 100 mg, about 125 mg, about 150 mg, about 200 mg, about 250 mg, about 300 mg, about 400 mg, or about 500 mg of Compound 1.

In another embodiment, the present disclosure provides a solid oral dosage form comprising a Composition of the Disclosure further comprising an exterior coating. In another embodiment, the exterior coating comprises a glidant. In another embodiment, the glidant is talc. In another embodiment, the exterior coating comprises a coating agent, glidant, a pigment, and a colorant.

In another embodiment, the present disclosure provides a method of treating a patient in need thereof, the method comprising administering to the patient a therapeutically effective amount of a Composition of the Disclosure, wherein the patient has cancer. In another embodiment, the cancer has a BRAF gene mutation, a NRAS gene mutation, or a BRAF gene mutation and a NRAS gene mutation. In another embodiment, the cancer has a BRAF gene mutation. In another embodiment, the cancer has a V600 BRAF gene mutation. In another embodiment, the cancer has a NRAS gene mutation. In another embodiment, the cancer is selected from the group consisting of skin cancer, ocular cancer, gastrointestinal cancer, thyroid cancer, breast cancer, ovarian cancer, lung cancer, brain cancer, laryngeal cancer, cervical cancer, lymphatic cancer, genitourinary cancer, and bone cancer.

In another embodiment, the present disclosure provides a method of treating a patient in need thereof, the method comprising administering to the patient a therapeutically effective amount of a Composition of the Disclosure, wherein the patient has cancer, and cells of the patient contain a biomarker. In another embodiment, the biomarker is a BRAF gene mutation, a NRAS gene mutation, or a BRAF gene mutation and a NRAS gene mutation. In another embodiment, the cancer is selected from the group consisting of skin cancer, ocular cancer, gastrointestinal cancer, thyroid cancer, breast cancer, ovarian cancer, lung cancer, brain cancer, laryngeal cancer, cervical cancer, lymphatic cancer, genitourinary cancer, and bone cancer.

In another embodiment, the present disclosure provides a kit comprising a Composition of the Disclosure, and instructions for administering the Composition of the Disclosure to a patient having cancer.

In another embodiment, the disclosure provides procedures of personalized medicine for patients having cancer, and encompasses the selection of treatment options with the highest likelihood of successful outcome for individual cancer patients. In another aspect, the disclosure relates to the use of an assay(s) to predict the treatment outcome, e.g., the likelihood of favorable responses or treatment success, in patients having cancer.

In another embodiment, the disclosure provides methods of selecting a patient, e.g., human subject, for treatment of cancer with a Composition of the Disclosure, comprising obtaining a biological sample, e.g., blood cells, from the patient, testing a biological sample from the patient for the presence of a biomarker, and selecting the patient for treatment if the biological sample contains the biomarker. In another embodiment, the methods further comprise administering a therapeutically effective amount of a Composition of the Disclosure to the patient if the biological sample contains the biomarker. Examples of biomarkers include, but are not limited to, BRAF mutation status and/or NRAS mutation status.

In another embodiment, the disclosure provides methods predicting treatment outcomes in a patient having cancer, comprising obtaining a biological sample from the patient, testing the biological sample from the patient for the presence of a biomarker, e.g., a BRAF mutation and/or a NRAS mutation, wherein the detection of the biomarker indicates the patient will respond favorably to administration of a therapeutically effective amount of a Composition of the Disclosure.

In another embodiment, the disclosure provides methods treating cancer, comprising administering a therapeutically effective amount of a Composition of the Disclosure to a patient, e.g., a human subject, with cancer in whom the patient's cells contain a biomarker, e.g., a BRAF mutation and/or a NRAS mutation. In one embodiment, the patient is selected for treatment with a Composition of the Disclosure after the patient's cells have been determined to contain a biomarker.

In another embodiment, the method of treating a patient having cancer comprises obtaining a biological sample from the patient, determining whether the biological sample contains a BRAF mutation and/or a NRAS mutation, and administering to the patient a therapeutically effective amount a Composition of the Disclosure, if the biological sample contains a BRAF mutation and/or a NRAS mutation.

The present disclosure provides the following particular embodiments with respect to personalized medicine for patients having cancer:

Embodiment I: A method of treating a patient having cancer, the method comprising administering a therapeutically effective amount of a Composition of the Disclosure to the patient, wherein cells of the patient contain a biomarker, and the biomarker is BRAF mutation status and/or NRAS mutation status.

Embodiment II: A method of treating a patient having cancer, the method comprising:

(a) determining the mutation status of BRAF and/or NRAS, in a biological sample from the patient, and when a BRAF and/or NRAS mutation is detected,

(b) administering to the patient a therapeutically effective amount of a Composition of the Disclosure.

Embodiment III: A method for treating a cancer in a patient having a BRAF and/or NRAS mutation, the method comprising administering to the patient a therapeutically effective amount of a Composition of the Disclosure.

Embodiment IV: The method of any one of Embodiments I-III, wherein at least one additional anticancer agent is administered to the patient.

Embodiment V: A method of treating a human patient having cancer, the method comprising:

(a) obtaining a biological sample from the patient;

(b) determining whether to biological sample has a BRAF and/or NRAS mutation; and

(c) administering to the patient a therapeutically effective amount a Composition of the Disclosure if the biological sample indicates a BRAF and/or NRAS mutation.

### Definitions

The term “Compound 1” as used herein refers to (R)-2-(1-(6-amino-5-chloropyrimidine-4-carboxamido)ethyl)-N-(5-chloro-4-(trifluoromethyl)pyridin-2-yl)thiazole-5-carboxamide. This compound is also known as MLN2480 and TAK580. The chemical structure of Compound 1 is:

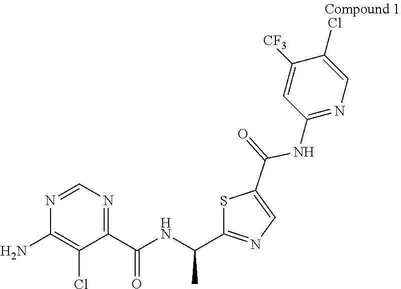 [SMILES: C[C@@H](NC(=O)c1ncnc(N)c1Cl)c1ncc(C(=O)Nc2cc(C(F)(F)F)c(Cl)cn2)s1]

The term “solid dispersion” as used herein refers to an amorphous dispersion comprising Compound 1 and a vinylpyrrolidone-vinyl acetate copolymer in a solid state that is prepared by hot melt extrusion.

The term “amorphous” as used herein refers to a solid form of Compound 1 or a solid dispersion comprising a solid form of Compound 1 that lacks the long-range order characteristic of a crystal, i.e., the solid is non-crystalline.

The terms “micronization,” “micronizing,” or “milling” as used herein refer to a process or method by which the size of a population of particles is reduced, typically to the micron scale.

The term “micron” or “μm” as used herein refer to “micrometer,” which is 1×10⁻⁶ meter.

The term “therapeutically effective amount” as used herein refers to the amount of Compound 1 sufficient to treat one or more symptoms of cancer, or cause regression of the cancer. For example, in one embodiment, a therapeutically effective amount will refer to the amount of Compound 1 that decreases the rate of tumor growth, decreases tumor mass, decreases the number of metastases, increases time to tumor progression, or increases survival time by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

The terms “a” and “an” refer to one or more than one.

The term “about,” as used herein, includes the recited number ±10%. Thus, “about 10” means 9 to 11.

As used herein, the term “particle size distribution” or “PSD” describes a stratification of particles in a powder dispersion according to the relative amount by mass of particles present in a specified size range. For example, in Table 8, 4.64 wt % of the powder dispersion has a particle diameter less than 45 microns, 25.50 wt % has a particle diameter between 45 and 75 microns, and so on. PSD can be measured by sieving with a woven sieve cloth or similar material. PSD can also be measured by laser diffraction using Malvern Master Sizer Microplus equipment or its equivalent, or other suitable techniques.

As used herein, the term “mass median diameter” or “D₅₀” describes the diameter where 50 mass-% of the particles in a powder dispersion have a larger equivalent diameter, and the other 50 mass-% have a smaller equivalent diameter as determined by laser diffraction in Malvern Master Sizer Microplus equipment or its equivalent, or other suitable techniques. For example, if the D₅₀ of a powder dispersion is 105 μm, then 50% of the particles are larger than 105 μm, and 50% of the particles are smaller than 105 μm. Likewise, the term “D₉₀” describes the diameter where 90 mass-% of the particles in a powder dispersion have a smaller equivalent diameter, and the other 10 mass-% have a larger equivalent diameter. The term “D₁₀” describes the diameter where 10 mass-% of the particles in a powder dispersion have a smaller equivalent diameter, and the other 90 mass-% have a larger equivalent diameter.

The term “patient” as used herein refers to a human having cancer.

The term “tablet” or “core tablet” as used herein refers to a tablet that does not have a film coating.

The term “film-coated tablet” or “FC tablet” as used herein refers to a tablet that has a film coating. In one embodiment, the coating is polymer-based.

“Crospovidone” is a cross-linked homopolymer of vinyl pyrrolidone (VP). One brand of crospovidone is Polyplasdone® XL-10.

The term “vinylpyrrolidone-vinyl acetate copolymer” as used herein refers a polymer comprising vinylpyrrolidone and vinyl acetate. Names and abbreviations for vinylpyrrolidone-vinyl acetate copolymer include, but are not limited to, copovidone, copovidonum, copolyvidone, copovidon, PVP-VAc-Copolymer. Copovidone is a vinylpyrrolidone-vinyl acetate copolymer comprised of 6 parts of vinylpyrrolidone and 4 parts of vinyl acetate e.g., CAS 25086-89-9. Examples of copovidone commercial products are Kollidon® VA 64 and Kollidon® 64 Fine. Another example is “Plasdone S-630,” a 60:40 random copolymer of N-vinyl pyrrolidinone and vinyl acetate.

“HPMCAS” refers to Hypromellose acetate succinate, a polymer containing acetyl and succinoyl groups. There are different types and grades of HPMCAS (e.g., HPMCAS-LG, HPMCAS-MG, HPMCAS-HG), which dissolve at different pHs due to different composition and ratio of its functional groups (e.g., acetyl, succinoyl).

“Eudragit® EPO” is a cationic copolymer based on dimethylaminoethyl methacrylate, butyl methacrylate, and methyl methacrylate.

“HPMCP” refers to a hydroxypropyl methylcellulose phthalate polymer. There are different types and grades of HPMCP (e.g., HP-55s, HP-50, HP-55), which dissolve at different pHs due to different composition and ratio of its functional groups (e.g., phthalyl).

“HPC” refers to hydroxypropyl cellulose. There are different types and grades of HPC (e.g., BPC-SSL, HPC-SL, HOC-SLT).

“POVACOAT®” refers to polyvinyl alcohol-acrylic acid-methacrylate copolymer. There are different types and grades of POVACOAT (e.g., Type MP, Type F, Type R) depending on the average molecule weight or average particle diameter.

“Hypromellose TC-5E” refers to hydroxypropyl methyl cellulose.

“Soluplus®” refers to polyvinyl caprolactam-polyvinyl acetate-polyethylene glycol graft copolymer.

The term “w/w” means by weight. For example, 50% w/w means that the mass of the substance is 50% of the total mass of the solution or mixture.

The term “pharmaceutically acceptable salt” as used herein refers to those salts suitable for use in contact with the tissues of humans without undue toxicity, irritation, allergic response and the like, and are commensurate with a reasonable benefit/risk ratio. Pharmaceutically acceptable salts are well known in the art. See Berge et al., J. Pharmaceutical Sciences, 1977, 66, 1-19.

The term “BRAF” as used herein refers to B-Raf proto-oncogene, serine/threonine kinase. BRAF functions as a serine/threonine kinase, has a role in regulating the MAP kinase/ERKs signaling pathway and can be found on chromosome 7q.

The term “NRAS” as used herein refers to neuroblastoma RAS viral (v-ras) oncogene homolog. NRAS functions as an oncogene with GTPase activity and can be found on chromosome 1p. NRAS interacts with the cell membrane and various effector proteins, such as Raf and RhoA, which carry out its signaling function through the cytoskeleton and effects on cell adhesion (Fotiadou et al. (2007) Mol. Gel. Biol. 27:6742-6755).

As used herein, the phrase “BRAF positive cancer,” “BRAF mutation-positive cancer,” “BRAF positive-mutation cancer,” or “BRAF positive-mutation cancer” means the cancer has one or more mutations in BRAF gene.

As used herein “NRAS positive cancer,” “NRAS mutation-positive cancer,” “NRAS positive-mutated cancer,” or “NRAS positive mutation cancer” means the cancer has one or more mutations in NRAS gene.

In some embodiments of the disclosure, the cancer is BRAF wild type and has one or more mutations in NRAS gene.

In some embodiments of the disclosure, the cancer is NRAS wild type and has one or more mutations in BRAF gene.

In some embodiments of the disclosure, the cancer has one or more mutations in both BRAF gene and NRAS gene.

The term “biomarker” as used herein refers to any biological compound, such as a protein, a fragment of a protein, a peptide, a polypeptide, a nucleic acid, etc. that can be detected and/or quantified in a patient in vivo or in a biological sample obtained from a patient. Furthermore, a biomarker can be the entire intact molecule, or it can be a portion or fragment thereof. In one embodiment, the expression level of the biomarker is measured. The expression level of the biomarker can be measured, for example, by detecting the protein or RNA (e.g., mRNA) level of the biomarker. In some embodiments, portions or fragments of biomarkers can be detected or measured, for example, by an antibody or other specific binding agent. In some embodiments, a measurable aspect of the biomarker is associated with a given state of the patient, such as a particular stage of cancer. For biomarkers that are detected at the protein or RNA level, such measurable aspects may include, for example, the presence, absence, or concentration (i.e., expression level) of the biomarker in a patient, or biological sample obtained from the patient. For biomarkers that are detected at the nucleic acid level, such measurable aspects may include, for example, allelic versions of the biomarker or type, rate, and/or degree of mutation of the biomarker, also referred to herein as mutation status.

For biomarkers that are detected based on expression level of protein or RNA, expression level measured between different phenotypic statuses can be considered different, for example, if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes.

In one embodiment, the biomarker is BRAF mutation status. In another embodiment, the measurable aspect of the BRAF mutation status is whether the BRAF gene contains at least one mutation.

In another embodiment, the BRAF mutation is V600 mutation. In another embodiment, the V600 mutation is V600E, V600G, V600A, or V600K; V600E, V600D, or V600K; or V600E, V600D, V600M, V600G, V600A, V600R, or V600K. In another embodiment, the BRAF mutation is V600E. In another embodiment, the BRAF mutation is V600D. In another embodiment, the BRAF mutation is V600K

The term “V600E mutation” means substitution of glutamic acid for valine at the amino acid position of 600. The term “V600K mutation” means substitution of lysine for valine at the amino acid position of 600. The term “V600D mutation” means substitution of aspartic acid for valine at the amino acid position of 600. The term “V600G mutation” means substitution of glycine for valine at the amino acid position of 600. The term “V600A mutation” means substitution of alanine for valine at the amino acid position of 600. The term “V600M mutation” means substitution of methionine for valine at the amino acid position of 600. The term “V600R mutation” means substitution of arginine for valine at the amino acid position of 600.

In another embodiment, the BRAF mutation is non-V600E mutation. In another embodiment the non-V600E mutation is G466A, G466V, N581S, D594H, R146W, L613F, D565_splice, S394*, P367R, G469A, G469V, G469*, G466V, G464V, G397S, S1131, A762E, G469L, D594N, G596S, G596R, D594N, D594H, or G327_splice. In one aspect, one or more non-V600E mutations are G469R, R95T, A621_splice, V639I, Q609H, G464V, or G466V. The asterisk “*” means a stop codon.

In another embodiment, the biomarker is NRAS mutation status. In another embodiment, the measurable aspect of the NRAS mutation status is whether the NRAS gene contains at least one mutation.

In another embodiment, the NRAS mutation is Q61R, Q61K, Q61L, Q61H, or Q61P. In one aspect, NRAS mutation is Q61R.

Thus, in certain aspects of the disclosure; the biomarker BRAF mutation status and/or NRAS mutation status which is differentially present in a subject of one phenotypic status (e.g., a patient having cancer with mutation of the BRAF gene) as compared with another phenotypic status (e.g., a normal undiseased patient or a patient having cancer without mutation of the BRAF gene).

In addition to individual biological compounds, e.g., BRAF or NRAS, the term “biomarker” as used herein is meant to include groups or sets of multiple biological compounds. For example, the combination of BRAF and NRAS may comprise a biomarker. Thus, a “biomarker” may comprise one, two, three, four, five, six, seven, eight, nine, ten, fifteen, twenty, twenty five, thirty, or more, biological compounds.

The determination of the expression level or mutation status of a biomarker in a patient can be performed using any of the many methods known in the art. In some embodiments, a mutation in a biomarker can be identified by sequencing a nucleic acid, e.g., a DNA, RNA, cDNA or a protein correlated with the marker gene, e.g., a genotype marker gene, e.g., BRAF or NRAS. There are several sequencing methods known in the art to sequence nucleic acids. A nucleic acid primer can be designed to bind to a region comprising a potential mutation site or can be designed to complement the mutated sequence rather than the wild type sequence. Primer pairs can be designed to bracket a region comprising a potential mutation in a marker gene. A primer or primer pair can be used for sequencing one or both strands of DNA corresponding to the marker gene. A primer can be used in conjunction with a probe, e.g., a nucleic acid probe, e.g., a hybridization probe, to amplify a region of interest prior to sequencing to boost sequence amounts for detection of a mutation in a marker gene. Examples of regions which can be sequenced include an entire gene, transcripts of the gene and a fragment of the gene or the transcript, e.g., one or more of exons or untranslated regions or a portion of a marker comprising a mutation site. Examples of mutations to target for primer selection and sequence or composition analysis can be found in public databases which collect mutation information, such as Database of Genotypes and Phenotypes (dbGaP) maintained by the National Center for Biotechnology Information (Bethesda, Md.) and Catalogue of Somatic Mutations in Cancer (COSMIC) database maintained by the Wellcome Trust Sanger Institute (Cambridge, UK).

Sequencing methods are known to one skilled in the art. Examples of methods include the Sanger method, the SEQUENOM™ method and Next Generation Sequencing (NGS) methods. The Sanger method, comprising using electrophoresis, e.g., capillary electrophoresis to separate primer-elongated labeled DNA fragments, can be automated for high-throughput applications. The primer extension sequencing can be performed after PCR amplification of regions of interest. Software can assist with sequence base calling and with mutation identification. SEQUENOM™ MASSARRAY® sequencing analysis (San Diego, Calif.) is a mass-spectrometry method which compares actual mass to expected mass of particular fragments of interest to identify mutations. NGS technology (also called “massively parallel sequencing” and “second generation sequencing”) in general provides for much higher throughput than previous methods and uses a variety of approaches (reviewed in Zhang et al. (2011) J. Genet. Genomics 38:95-109 and Shendure and Hanlee (2008) Nature Biotech. 26:1135-1145). NGS methods can identify low frequency mutations in a marker in a sample. Some NGS methods (see, e.g., GS-FLX Genome Sequencer (Roche Applied Science, Branford, Conn.), Genome analyzer (Illumina, Inc. San Diego, Calif.) SOLID™ analyzer (Applied Biosystems, Carlsbad, Calif.), Polonator G.007 (Dover Systems, Salem, N.H.), HELISCOPE™ (Helicos Biosciences Corp., Cambridge, Mass.) use cyclic array sequencing, with or without clonal amplification of PCR products spatially separated in a flow cell and various schemes to detect the labeled modified nucleotide that is incorporated by the sequencing enzyme (e.g., polymerase or ligase). In one NGS method, primer pairs can be used in PCR reactions to amplify regions of interest. Amplified regions can be ligated into a concatenated product. Clonal libraries are generated in the flow cell from the PCR or ligated products and further amplified (“bridge” or “cluster” PCR) for single-end sequencing as the polymerase adds a labeled, reversibly terminated base that is imaged in one of four channels, depending on the identity of the labeled base and then removed for the next cycle. Software can aid in the comparison to genomic sequences to identify mutations. Another NGS method is exome sequencing, which focuses on sequencing exons of all genes in the genome. As with other NGS methods, exons can be enriched by capture methods or amplification methods.

In some embodiments, DNA, e.g., genomic DNA corresponding to the wild type or mutated marker can be analyzed both by in situ and by in vitro formats in a biological sample using methods known in the art. DNA can be directly isolated from the sample or isolated after isolating another cellular component, e.g., RNA or protein. Kits are available for DNA isolation, e.g., QIAAMP® DNA Micro Kit (Qiagen, Valencia, Calif.). DNA also can be amplified using such kits.

In another embodiment, mRNA corresponding to the marker can be analyzed both by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). RNA can be isolated using standard procedures (see e.g., Chomczynski and Sacchi (1987) Anal. Biochem. 162: 156-159), solutions (e.g., trizol, TRI REAGENT® (Molecular Research Center, Inc., Cincinnati, Ohio; see U.S. Pat. No. 5,346,994) or kits (e.g., a QIAGEN® Group RNEASY® isolation kit (Valencia, Calif.) or LEUKOLOCK™ Total RNA Isolation System, Ambion division of Applied Biosystems, Austin, Tex.).

Additional steps may be employed to remove DNA from RNA samples. Cell lysis can be accomplished with a nonionic detergent, followed by microcentrifugation to remove the nuclei and hence the bulk of the cellular DNA. DNA subsequently can be isolated from the nuclei for DNA analysis. In one embodiment, RNA is extracted from cells of the various types of interest using guanidinium thiocyanate lysis followed by CsCl centrifugation to separate the RNA from DNA (Chirgwin et al. (1979) Biochemistry 18:5294-99). Poly(A)+RNA is selected by selection with oligo-dT cellulose (see Sambrook et al. (1989) Molecular Cloning—A Laboratory Manual (2nd ed.), Cold Spring Harbor Laboratory, Cold Spring Harbor, N.Y.). Alternatively, separation of RNA from DNA can be accomplished by organic extraction, for example, with hot phenol or phenol/chloroform/isoamyl alcohol. If desired, RNAse inhibitors may be added to the lysis buffer. Likewise, for certain cell types, it may be desirable to add a protein denaturation/digestion step to the protocol. For many applications, it is desirable to enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX® medium (see Ausubel et al. (1994) Current Protocols In Molecular Biology, vol. 2, Current Protocols Publishing, New York). Once bound, poly(A)+mRNA is eluted from the affinity column using 2 mM EDTA/0.1% SDS.

A characteristic of a biomarker of the invention in a sample, e.g., after obtaining a sample (e.g., a tumor biopsy) from a test subject, can be assessed by any of a wide variety of well known methods for detecting or measuring the characteristic, e.g., of a marker or plurality of markers, e.g., of a nucleic acid (e.g., RNA, mRNA, genomic DNA, or cDNA) and/or translated protein. Non-limiting examples of such methods include immunological methods for detection of secreted, cell-surface, cytoplasmic, or nuclear proteins, protein purification methods, protein function or activity assays, nucleic acid hybridization methods, optionally including “mismatch cleavage” steps (Myers, et al. (1985) Science 230:1242) to digest mismatched, i.e. mutant or variant, regions and separation and identification of the mutant or variant from the resulting digested fragments, nucleic acid reverse transcription methods, and nucleic acid amplification methods and analysis of amplified products. These methods include gene array/chip technology, RT-PCR, TAQMAN® gene expression assays (Applied Biosystems, Foster City, Calif.), e.g., under GLP approved laboratory conditions, in situ hybridization, immunohistochemistry, immunoblotting, FISH (fluorescence in situ hybridization), FACS analyses, northern blot, southern blot, INFINIUM® DNA analysis Bead Chips (Illumina, Inc., San Diego, Calif.), quantitative PCR, bacterial artificial chromosome arrays, single nucleotide polymorphism (SNP) arrays (Affymetrix, Santa Clara, Calif.) or cytogenetic analyses.

Examples of techniques for detecting differences of at least one nucleotide between two nucleic acids include, but are not limited to, selective oligonucleotide hybridization, selective amplification, or selective primer extension. For example, oligonucleotide probes can be prepared in which the known polymorphic nucleotide is placed centrally (allele- or mutant-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques can be used for the simultaneous detection of several nucleotide changes in different polymorphic or mutated regions of NRAS. For example, oligonucleotides having nucleotide sequences of specific allelic variants or mutants are attached to a solid support, e.g., a hybridizing membrane and this support, e.g., membrane, is then hybridized with labeled sample nucleic acid. Analysis of the hybridization signal thus can reveal the identity of the nucleotides of the sample nucleic acid.

The term “pharmaceutically acceptable excipient” or “excipient” as used herein refers to any ingredient in a Composition of the Disclosure other than the solid dispersion of Compound 1 and vinylpyrrolidone-vinyl acetate copolymer. An excipient is typically an inert substance added to a composition to facilitate processing, handling, administration, etc. of the composition. Useful excipients include, but are not limited to, adjuvants, antiadherents, binders, carriers, disintegrants, fillers, flavors, colors, diluents, lubricants, glidants, preservatives, sorbents, solvents, surfactants, and sweeteners.

Conventional pharmaceutical excipients are well known to those of skill in the art. In particular, one of skill in the art will recognize that a wide variety of pharmaceutically acceptable excipients can be used in admixture with the solid dispersion of Compound 1 and vinylpyrrolidone-vinyl acetate copolymer, including those listed in the Handbook of Pharmaceutical Excipients, Pharmaceutical Press 4th Ed. (2003), and Remington: The Science and Practice of Pharmacy, Lippincott Williams & Wilkins, 21st ed. (2005).

### EXAMPLES

### Example 1

### Screening of Solid Dispersions by DSC and Oil Bath Methods

Solid dispersions made by a hot melt extrusion process are referred to as a hot melt extrudate or “HME” in the EXAMPLES and Figures.

Differential Scanning calorimetry (DSC) and Oil bath methods were applied to study solid dispersions comprising Compound 1. DSC is applied for the prediction of miscibility and solubility of Compound 1 in polymer.

### Materials

Hypromellose phthalate HPMCP (55, 55s, 50), Hypromellose acetate succinate HPMC-AS (LG, MG, HG) and Hypromellose TC-5E were obtained from Shin-Etsu. Eudragit EPO was obtained by Evonik. HPC-SSL was obtained from Nippon Soda. Kollidon VA64 and Soluplus were obtained from BASF. POVACOAT TypeMP was obtained from Daido Chemical Corporation.

### DSC Method and Evaluation

Pure crystalline drug Compound 1 was physically mixed with each pure polymer. The equilibrium solution temperature (Tend) and Enthalpy (H) of Compound 1 in each 20% Compound 1 loaded physical mixture (PM) were measured for polymer screening by DSC (Discovery™ DSC, TA Instruments) with following steps;

To 105° C.

Keep 105° C. in 10 min

105° C. to −20° C. @-10° C./min

−20° C. to over MP (melting point) of Compound 1 (206° C.) @1° C./min

From the endothermic peak of DSC, H and Tend were analyzed, and the change ratio A E between pure Compound 1 and PM was defined as miscibility parameters (Eq. 1). Generally, the PM which shows higher miscibility with Compound 1 makes the Compound 1 endothermic peak shifted to lower temperature and the peak intensity become smaller. Therefore, the lower A E means the higher miscibility.

$$Δ E = \frac{{H}_{p m}}{{H}_{A}} + \frac{{T}_{end , p m}}{{T}_{end , A}} \qquad Eq . 1$$

$$( A : Compound 1 , p m : physical mixture )$$

### Oil Bath Method and Evaluation

Compound 1 was physically mixed with each polymer (Compound 1 ratio in polymer=20% w/w for polymer screening, 40, 50, and 60% w/w for loading amount screening), Approx. 100-500 mg of PM was put into a glass tube and melt and mixed in the tube with spatula while heating in Oil bath (180-200° C.). The visual appearance and Endotherm peak by mDSC (Modulated Differential Scanning calorimetry) were evaluated for polymer screening, and the chemical and physical properties were evaluated for loading amount screening.

### Results of Polymer Screening by DSC and Oil Bath

Results summary of polymer screening by DSC and Oil bath methods are shown in Table 1. PM with HPMC-AS, Kollidon VA64 and Soluplus showed relatively lower A E by DSC, and all these Oil bath materials showed amorphous (clear appearance and no API endothermic peak by mDSC).

TABLE 1
Results summary of polymer screening by DSC and Oil bath methods

| | | DSC | | | Oil bath | |
|---|---|---|---|---|---|---|
| No. | Polymer | H [J/g] | T_{end} [° C.] | ΔE [—] | Appear- ance | mDSC |
| 1 | HPMCAS-LG | 7.359 | 187.221 | 0.96 | Clear | no peak |
| 2 | HPMCAS-MG | 9.052 | 177.449 | 0.93 | Clear | no peak |
| 3 | HPMCAS-HG | 7.828 | 174.315 | 0.91 | Clear | no peak |
| 4 | EUDRAGIT EPO | 16.092 | 176.173 | 1.00 | Unclear | API peak |
| 5 | HPMCP (HP-55s) | 11.261 | 181.525 | 1.03 | Unclear | API peak |
| 6 | HPC-SLT | 15.871 | 174.488 | 1.10 | Clear | API peak |
| 7 | HPMCP (HP-50) | 15.6 | 184.786 | 0.97 | Unclear | API peak |
| 8 | HPMCP (HP-55) | 26.391 | 175.883 | 0.99 | Unclear | API peak |
| 9 | Kollidon VA64 | 8.136 | 170.273 | 0.89 | Clear | no peak |
| 10 | POVACOAT TypeMP | 36.971 | 200.41 | 1.32 | Unclear | API peak |
| 11 | Soluplus | 7.802 | 167.679 | 0.87 | Clear | no peak |
| 12 | TC-5E | 11.802 | 193.887 | 1.04 | Unclear | API peak |
| 13 | Compound 1 | 102.52 | 209.958 | 2.00 | — | — |

TABLE 2
Results summary of API loading amount screening in HME by Oil bath method

| | Com- | % Compound 1 in HME by Oil bath | | |
|---|---|---|---|---|
| Test items | pound 1 | 40% | 50% | 60% |
| Appearance | — | Clear | Clear | Unclear |
| Assay [%] | 99.7* | 99.7 | 99.4 | 90.8 |
| Impurities | | | | |
| Individual [%] | <0.05* | <0.05 | <0.05 | <0.05 |
| Total [%] | <0.05* | <0.05 | <0.05 | <0.05 |
| Enantiomer [%] | 0.5* | 2.01 | 2.08 | 1.90 |
| mDSC Tg** onset [° C.] | — | 104.996 | 104.326 | 103.577 |
| | | no peak | no peak | API peak |
| Solubility (24 h, 37° C.) | — | | | |
| JP1 [ug/mL] | 3.4 | 59.5 | 52.7 | 68.6 |
| JP2 [ug/mL] | 1.1 | 21.3 | 20.0 | 19.0 |

*RoA (Results of analysis)
**Glass Transition Temperature

### Results of API Loading Amount Screening by Oil Bath Method

The results summary of Compound 1 loading amount screening by the Oil bath method is shown in Table 2. All samples from the Oil bath method showed higher solubility in both JP1 (pH 1.2) and JP2 (pH 6.8) than Compound 1 itself. 60% HME by Oil bath showed existence of crystalline API from both appearance and mDSC.

### Example 2

### Tablet Formulations

### HME Loading Amount Screening in Tablet

Tablets were manufactured with 40, 50, 60, and 70% solid dispersion loading amount using a solid dispersion comprising 40% Compound 1 and 60% Kollidon VA64 made using a hot melt extrusion process. This solid dispersion is referred to as HME (40%). The PSD of this HME (40%) on a 250 μm screen is shown in Table 3.

### Sample Preparation

HME (40%) was blended with MCC (Microcrystalline cellulose), croscarmellose sodium and colloidal silicon dioxide with mortar and pestle. And then the blended powder was put into a glass bottle with magnesium stearate and shake it gently for 100 times at 20 tabs scale. The blended powder was compressed on a single hand tablet press (HANDTAB-200, Ichihashi seiki) into tablets with various compression forces. The tablet properties and dissolution (CTAB concentration depends on Compound 1 loading amount in each tablet to keep same sink condition) were measured. The tablet formulations with different HME (40%) loading amount are shown in Table 4.

TABLE 3
HME (40%) by sieving

| Sieve size [μm] | >500 | 355-500 | 224-355 | 160-224 100-160 | | 71-100 | 50-71 | <45 |
|---|---|---|---|---|---|---|---|---|
| Frequency [%] | 0.0 | 0.0 | 0.9 | 16.1 | 80.4 | 2.2 | 0.6 | 0.0 |

TABLE 4
Tablet formulations for HME loading amount screening in tablet

| | 20150107-2 | | 20150213-1 | | 20150213-5 | |
|---|---|---|---|---|---|---|
| Lot No. | % (w/w) | mg/tab | % (w/w) | mg/tab | % (w/w) | mg/tab |
| HME (40%) | 50 | 312.5 | 50 | 312.5 | 50 | 312.5 |
| MCC (PH101) | 41 | 256.25 | | | | |
| MCC (UF702) | | | 41 | 256.25 | | |
| MCC (UF711) | | | | | 41 | 256.25 |
| Croscarmellose Sodium | 8 | 50 | 8 | 50 | 8 | 50 |
| Colloidal Silicon Dioxide | 0.5 | 3.125 | 0.5 | 3.125 | 0.5 | 3.125 |
| Magnesium Stearate | 0.5 | 3.125 | 0.5 | 3.125 | 0.5 | 3.125 |
| Total | 100 | 625 | 100 | 625 | 100 | 625 |

### Results

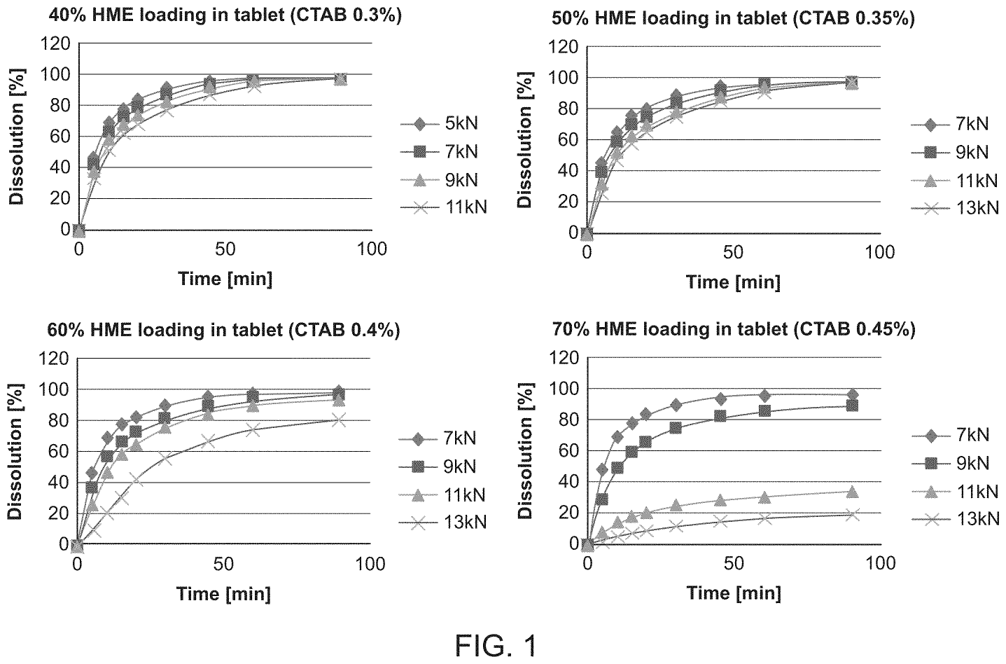

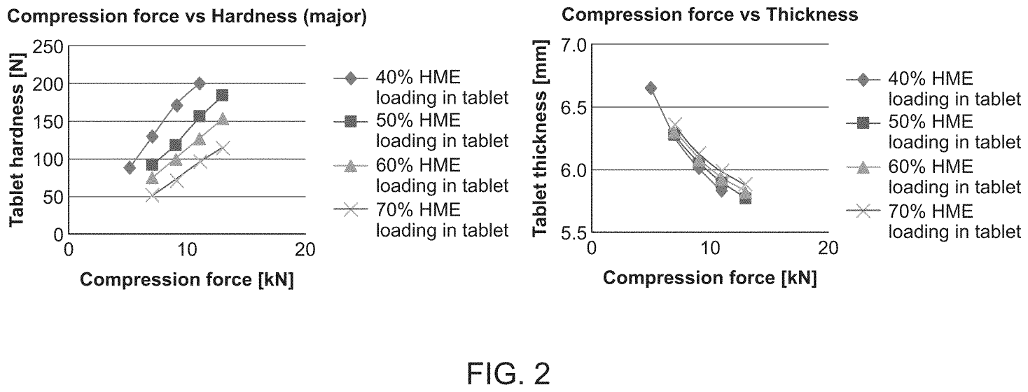

Dissolution profiles and tablet properties of each % HME loaded tablets are shown in FIG. 1 and FIG. 2. In FIG. 1, 60% and 70% HME loaded tablets showed slower dissolution profile at higher compression force due to the strong hydrogel-matrix formation in tablet. Furthermore, these two HME tablets showed lower hardness than the other two in FIG. 2, and the hardness did not reach 150 N which is a target hardness considering friability. All of 40% and 50% HME loaded tablets showed quick dissolution profile regardless of the compression force, and the hardness could reach 150 N by controlling compression force.

### Example 3

### Tablets

Prototype tablets were manufactured to select fillers for tablet formulation.

### Sample Preparation

HME (40%) was blended with various fillers (MCC, DCPA (dibasic calcium phosphate anhydrous), SDS (sodium dodecyl sulfate) and these combinations), croscarmellose sodium, colloidal silicon dioxide with mortar and pestle. And then the blended powder was put into a glass bottle with magnesium stearate and shake it gently for 100 times at 20 tabs scale. The blended powder was compressed on a single hand tablet press (HANDTAB-200, Ichihashi seiki) into tablets with various compression forces. And then, the tablet properties and dissolution were evaluated. HME loading amount in tablet formulation was fixed as 50%, and the dose strength of each tablet was 125 mg/tab in this study. The tablet formulations are shown in Table 5.

TABLE 5
Tablet formulations for main filler screening in tablet

| | 20150107-2 | | 20150213-1 | | 20150213-5 | |
|---|---|---|---|---|---|---|
| Lot No. | % (w/w) | mg/tab | % (w/w) | mg/tab | % (w/w) | mg/tab |
| HME (40%) | 50 | 312.5 | 50 | 312.5 | 50 | 312.5 |
| MCC (PH101) | 41 | 256.25 | | | | |
| MCC (UF702) | | | 41 | 256.25 | | |
| MCC (UF711) | | | | | 41 | 256.25 |
| Croscarmellose Sodium | 8 | 50 | 8 | 50 | 8 | 50 |
| Colloidal Silicon Dioxide | 0.5 | 3.125 | 0.5 | 3.125 | 0.5 | 3.125 |
| Magnesium Stearate | 0.5 | 3.125 | 0.5 | 3.125 | 0.5 | 3.125 |
| Total | 100 | 625 | 100 | 625 | 100 | 625 |

### Results

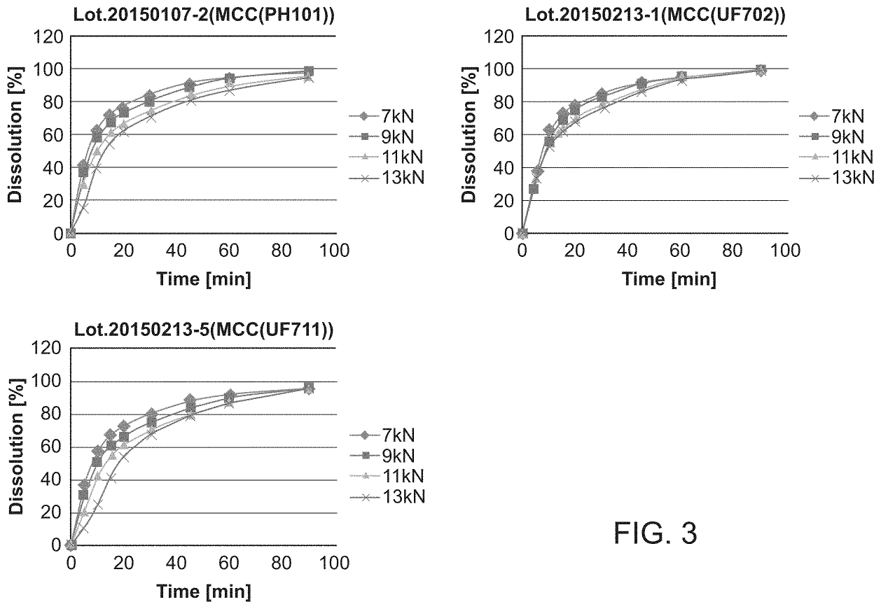

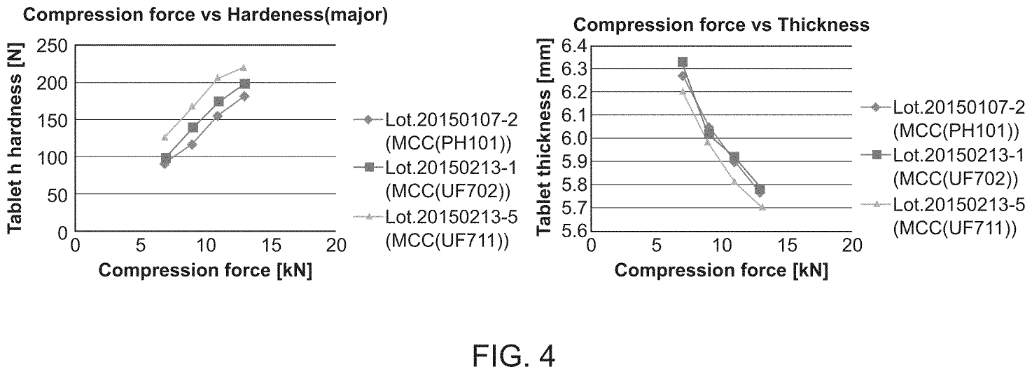

The dissolution profiles and tablet properties of the tablet formulations are shown in FIG. 3 and FIG. 4. From these results, MCC OF grade (CEOLUS, Asashi-Kasei), especially UF711 showed higher tablet hardness than PH101 (Avicel, FMC Biopolymer). All tablet formulations showed similar dissolution profiles regardless of MCC grade and their tablet hardness. UF711 was selected as the filler in a high dose strength tablet formulation.

### Example 4

### Feasibility Assessment of Prototype HME (50%) Formulation

A solid dispersion comprising 50% Compound 1 and 50% Kollidon VA64 was prepared using a hot melt extrusion process. This solid dispersion is referred to as prototype HME (50%).

### Solid Dispersion Powder Preparation by Mini-Extruder

Compound 1 was physically mixed with Kollidon VA64 (50% loading amount) and a HME strand was obtained using a Mini-Extruder (Hybrid 5/9 mm, Three Tech). The process conditions of Mini-Extruder for this study are shown in Table 6. After the extrusion process, the HME strand was manually milled by mortar and pestle to give prototype HME (50%). The chemical/physical properties and stability of prototype HME (50%) were evaluated. An impact of temperature conditions on solid dispersion quality was also checked.

TABLE 6
Process conditions of Mini-Extruder

| Heating zone number | 3 zones | |
|---|---|---|
| Temperature conditions | Lot No. | Heating zone temperature (H1, H2, H3) |
| | 20150406-01 | H1_130° C., H2_175° C., H3_175° C. |
| | 20150406-02 | H1_150° C., H2_185° C., H3_185° C. |
| | 20150507-1 | H1_140° C., H2_180° C., H3_180° C. |

| | |
|---|---|
| Screw diameter | 9 mm |
| Screw speed | 75 rpm |
| Die diameter | 1.0 mm |
| Feeding speed | Manual feed |

Tablet Preparation with Prototype Solid Dispersion Powder

50% HME loaded tablets (150 mg) were manufactured with prototype HME (50%) produced with Mini-Extruder (process conditions are in Table 6). The HME (50%) was blended with MCC (UF711), croscarmellose sodium, colloidal silicon dioxide with mortar and pestle. And then the blended powder was put into a glass bottle with magnesium stearate and shake it gently for 100 times at 20 tabs scale. The blended powder was compressed on a single hand tablet press (HANDTAB-200, Ichihashi seiki) into tablets with 16×9 mm, oval size, and then film coating was conducted by Mini-coater (Mini Coater/Drier-2, Caleva (FIG. 10)). The tablet formulation is shown in Table 7.

TABLE 7
50% HME loaded tablet formulation

| | 20150805 | |
|---|---|---|
| Lot No. | % (w/w) | mg/tab |
| Prototype HME (50%) (Lot. 20150107-1) | 50 | 300 |
| MCC (UF711) | 41 | 246 |
| Croscarmellose Sodium | 8.0 | 48 |
| Colloidal Silicon Dioxide | 0.5 | 3.0 |
| Magnesium Stearate | 0.5 | 3.0 |
| Sub-total | 100 | 600 |
| Opadry Yellow 03F42240 | 2.3 | 14 |
| Opadry Red 03F45081 | 1.2 | 7 |
| Total | | 621 |

### Results

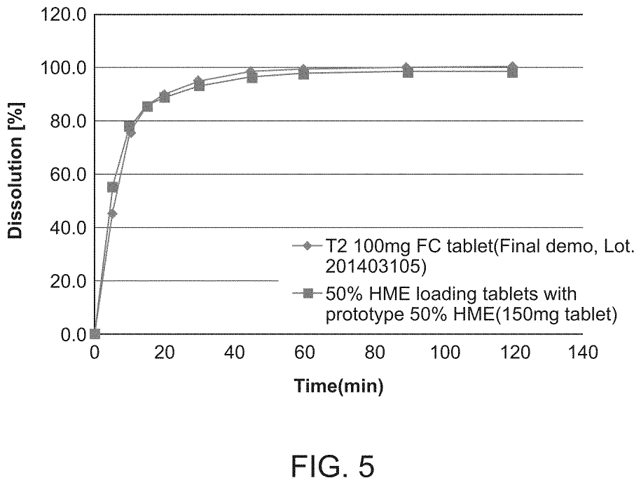

The PSD by sieving and the physicochemical properties of prototype HME (50%) are shown in Table 8 and Table 9. The chemical/physical properties and stability of prototype HME (50%) was comparable with HME (40%). A temperature dependent enantiomer increase was observed from the comparison among these three temperature conditions, but the enantiomer didn't increase during the stability. And the dissolution profiles of prototype 150 mg HME (50%) tablet (Table 8) and HME (40%) 100 mg film-coated (FC) tablet are shown in FIG. 5. Both tablets showed very similar dissolution profiles although the dose strengths were different.

TABLE 8
Prototype HME (50%) PSD by sieving

| | | | | | | | | |
|---|---|---|---|---|---|---|---|---|
| Sieve size | >500 | 355-500 | 250-355 | 180- | 106-160 | 75-100 | 45-75 | <45 |
| Frequency [%] | 0.99 | 0.33 | 1.32 | 17.55 | 28.81 | 20.86 | 25.50 | 4.64 |

TABLE 9
Results summary of prototype HME (50%)

| | Compound 1 | HME (40%) | Prototype HME (50%) | | |
|---|---|---|---|---|---|
| Lot No. | B26216-038-16 | 20140399 | 20150406-01 | 20150406-02 | 20150507-1 |
| Appearance | — | — | Clear | Clear | Clear |
| Assay [%] | 100.2 | 97.1 | 99.9 | 100.8 | 100.5 |
| Impurities | | | | | |
| Individual [%] | <0.05 | <0.05 | <0.05 | <0.05 | <0.05 |
| Total [%] | <0.05 | <0.05 | <0.05 | <0.05 | <0.05 |
| Enantiomer [%] | 0.20 | 0.87 | 1.33 | 4.71 | 1.68 |
| XRPD | — | — | no peak | no peak | no peak |
| mDSC Tg onset [° C.] | — | — | 105.0 | 106.7 | 106.7 |
| Solubility (24 h, 37° C.) | | | | | |
| JP1 [ug/mL] | — | 2.5 | 39.1 | 44.2 | 44.1 |
| JP2 [ug/mL] | — | 1.0 | 17.7 | 18.6 | 26.7 |

Stability @40° C./75% RH closed 1M

| | | | | | |
|---|---|---|---|---|---|
| Assay [%] | — | — | — | 100.5 | 98.8 |
| Impurities | | | | | |
| Individual [%] | — | — | — | <0.05 | <0.05 |
| Total [%] | — | — | — | <0.05 | <0.05 |
| Enantiomer [%] | — | — | — | 1.49 | 4.49 |
| XRPD | — | — | — | no peak | no peak |
| mDSC Tg onset [° C.] | — | — | — | 106.0 | 107.4 |

### Example 5

### Milling Study of HME

To find an optimal PSD, a milling study was conducted with several milling conditions using HME (40%) (Lot. 11122755). Tablets were prepared with each milled HME powder to check the dissolution and tablet properties.

### Sample Preparation

Milling was conducted with pin-mill (Nara sample mill SAM T, Nara machinery). Milling speed, screen size and milling rotor type were optimized. HME PSD was measured by sieving. Each milled HME powder with several conditions was blended with MCC (UF711), croscarmellose sodium, colloidal silicon dioxide with mortar and pestle. And then the blended powder was put into a glass bottle with magnesium stearate and shake it gently for 100 times at 30 tabs scale. The blended powder was compressed on a single hand tablet press (HANDTAB-200, Ichihashi seiki) into tablets with 16×9 mm, oval size with various compression forces. In this study, the impact of HME loading amount in tablet (50% and 60%) and MCC OF grade (UF702 and UF711) on the tablet property and dissolution were also checked. Milling conditions and tablet formulations in this study are shown in Table 10, Table 11 and Table 12.

TABLE 10
Milling conditions for milling study

| Milling | Screen size | Milling speed | Rotor |
|---|---|---|---|
| No. | [mm] | [rpm] | type |
| 2_1 | 0.7 | 8000 | |
| 2_2 | 0.7 | 10000 | |
| 2_3 | 0.7 | 12000 | |
| 3_1 | 0.8 | 8000 | pin rotor |
| 3_2 | 0.8 | 10000 | |
| 4_1 | 1.0 | 8000 | blade rotor |
| 4_2 | 1.0 | 10000 | |
| 4_3 | 1.0 | 12000 | |
| 5_1 | 0.6 | 8000 | |
| 5_2 | 0.6 | 10000 | |
| 6_1 | 0.7 | 8000 | |
| 6_2 | 0.7 | 10000 | |
| 7_2 | 0.8 | 10000 | |
| 8_1 | 1.0 | 8000 | pin rotor |
| 8_2 | 1.0 | 10000 | |
| HME (40%) | 0.5 | 10000 | |
| (Lot. 11122754) | | | |

TABLE 11
Tablet formulation and milling sample allocation
for milling study

| | Tablet | | |
|---|---|---|---|
| | formulation | | |
| | HME | | |
| | loading | | Milling |
| Formulation | amount | MCC | sample |
| No. | in Tablet | grade | No. |
| 1 | 50% | UF702 | 4_1 |
| 2 | 50% | UF711 | |
| 3 | 60% | UF702 | |
| 4 | 60% | UF711 | |
| 5 | 50% | UF702 | 4_2 |
| 6 | 50% | UF711 | |
| 7 | 60% | UF702 | |
| 9 | 50% | UF702 | 4 3 |
| 11 | 60% | UF702 | |
| 13 | 50% | UF702 | 5_1 |
| 14 | 50% | UF711 | |
| 15 | 60% | UF702 | |
| 17 | 50% | UF702 | 5_2 |
| 18 | 50% | UF711 | |
| 19 | 60% | UF702 | |
| 21 | 50% | UF702 | 6_1 |
| 22 | 50% | UF711 | |
| 23 | 60% | UF702 | |
| 24 | 60% | UF711 | |
| 25 | 50% | UF702 | 6_2 |
| 27 | 60% | UF702 | |
| 33 | 50% | UF702 | HME (40%) |
| 35 | 60% | UF702 | (Lot. 11122754) |

TABLE 12
Tablet formulation for milling study

| | 1, 5, 9, 13, 17, 21, | | 2, 6, 14 , 18, 22 | | 3, 7, 11, 15, 19, 23, | | 4 1, 24 | |
|---|---|---|---|---|---|---|---|---|
| Tablet formulation | % | mg/tab | % (w/w) | mg/tab | % (w/w) | mg/tab | % | mg/tab |
| HME (40%) | 50 | 312.5 | 50 | 312.5 | 60 | 375 | 60 | 375 |
| MCC (UF702) | 41 | 256.25 | — | — | 31 | 193.75 | — | — |
| MCC (UF711) | — | — | 41 | 256.25 | — | — | 31 | 193.75 |
| Croscarmellose | 8.0 | 50 | 8.0 | 50 | 8.0 | 50 | 8.0 | 50 |
| Sodium | | | | | | | | |
| Colloidal Silicon | 0.5 | 3.125 | 0.5 | 3.125 | 0.5 | 3.125 | 0.5 | 3.125 |
| Dioxide | | | | | | | | |
| Magnesium Stearate | 0.5 | 3.125 | 0.5 | 3.125 | 0.5 | 3.125 | 0.5 | 3.125 |
| Total | 100 | 625 | 100 | 625 | 100 | 625 | 100 | 625 |

### Results

HME PSD data by sieving for milled HME with various milling conditions are shown in Table 13. HME (40%) Lot. 11122754 represents a sample milled using the conditions described in WO 2015/148828.

Screen size, milling speed, and rotor type changes were effective parameters to optimize HME PSD. The blade rotor (New) worked better than pin rotor (Old) to obtain a narrow HME PSD and reduce fine HME particles (<75 μm) which has an impact on compression. On the other hand, larger screen size may increase the ratio of very large HME particles (>250 μm) which could cause slower dissolution speed.

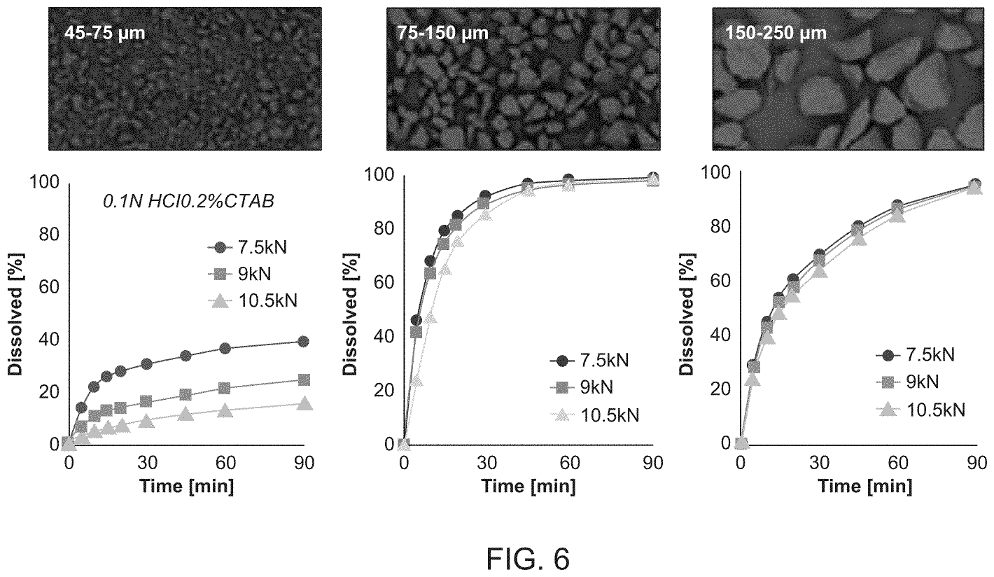

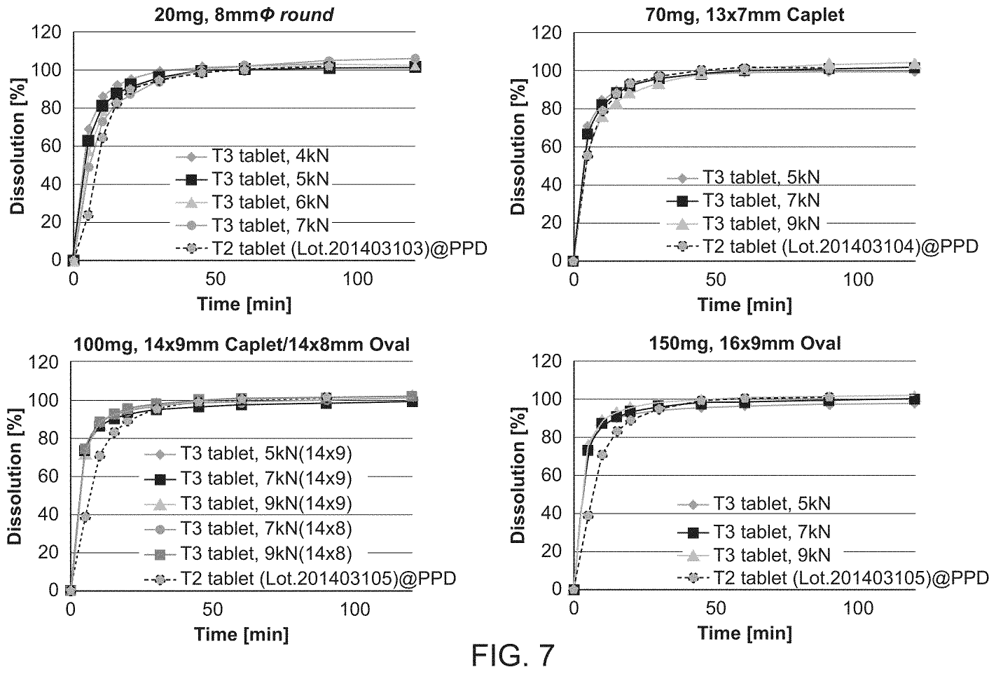

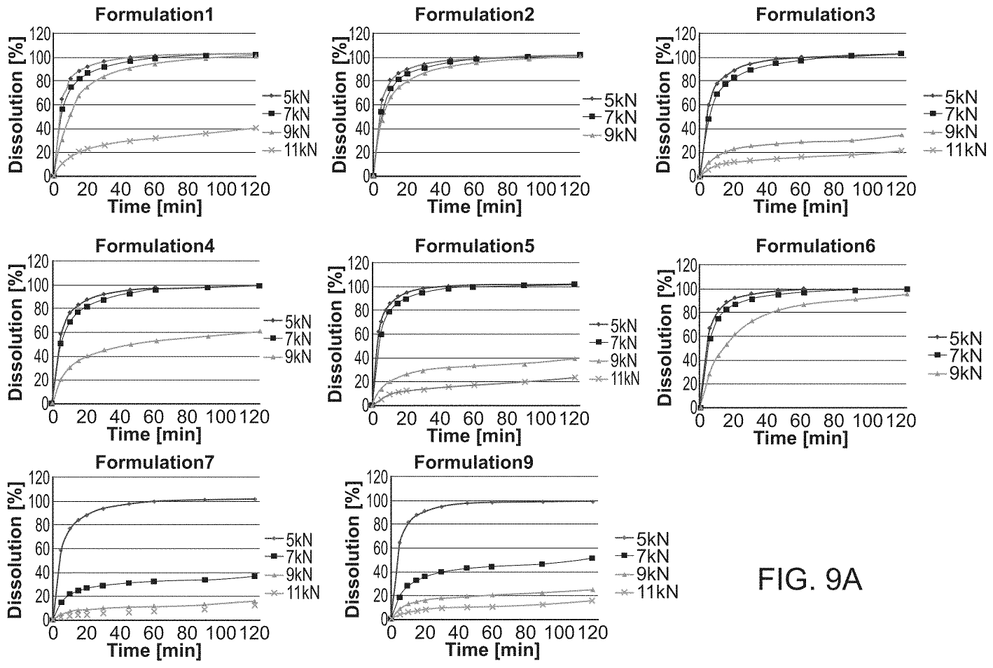

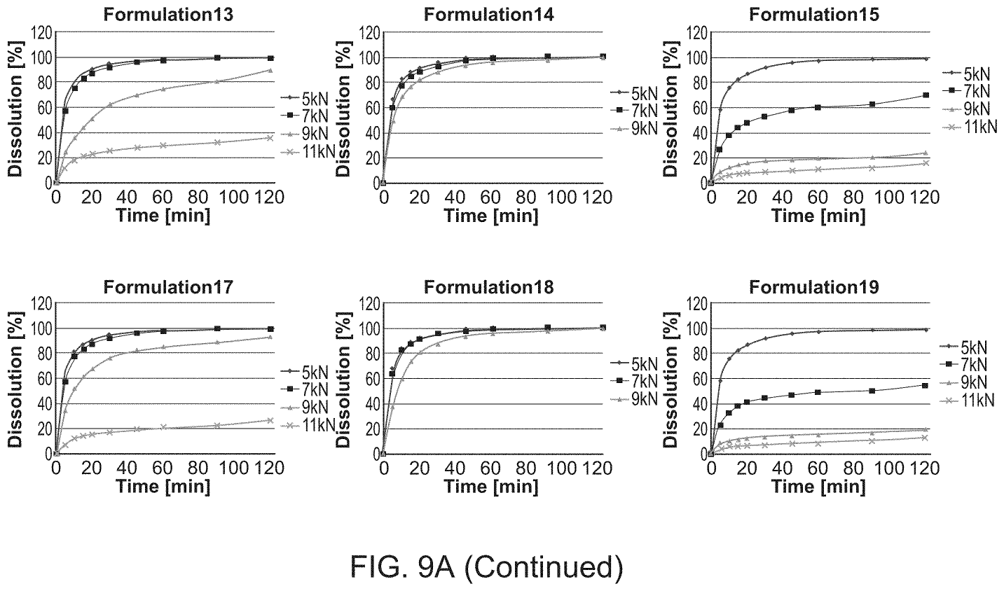

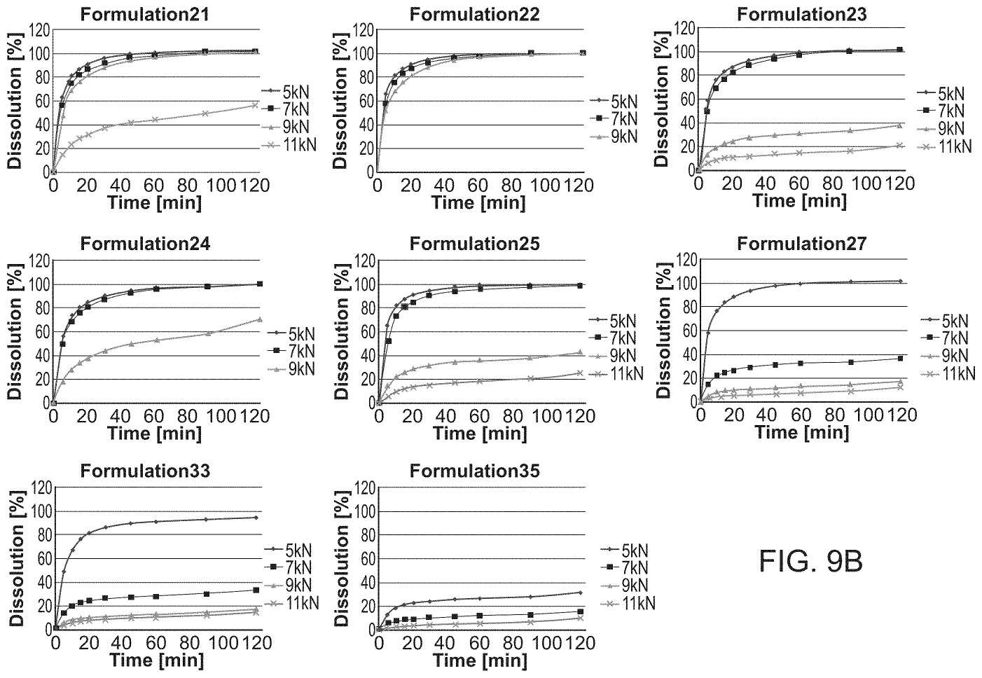

The summary of the milling study and individual dissolution profiles of tablets are shown in Table 14 and FIGS. 9A and 9B. PSDs of less than 75 μm and more than 250 μm have an impact on the dissolution profile (FIG. 6), therefore, HME PSD results were shown by 3 separated ranges, >250 μm, 75-250 μm and <75 μm. Minimum criteria were set for tablet hardness and dissolution to find good milling conditions. In Table 12, tablet formulation (2, 6, 14, 18, 22), 50% HME loaded tablet with MCC (UF711) showed quicker dissolution and higher tablet hardness than the other tablet formulations with 60% and/or MCC (UF702). From these dissolution and particle size of tablet formulation (9, 11, 33, 35), the content of fine particles (<75 μm) had an impact on dissolution, and ideally it should be less than 30% (Milling sample 4_1, 5_1˜6_2). Compared with the dissolution profiles of Formulation 33 and 35, the other Tablet formulations with a HME PSD having a smaller amount of fine particle improved the tablet hardness and dissolution.

TABLE 13
HME PSD results by sieving

| Milling No. | | | | | | | | | | | |
|---|---|---|---|---|---|---|---|---|---|---|---|
| (screen size | <45 pass | 45-75 | 75-106 | 106-180 | 180-250 | 250-355 | 355-500 | >500 | D₁₀* | D₅₀* | D₉₀* |
| [mm]/speed | [%] | [%] | [%] | [%] | [%] | [%] | [%] | [%] | [μm] | [μm] | [μm] |
| 2_1 (0.7/8000/old) | 1.62 | 16.25 | 17.12 | 33.61 | 19.05 | 10.3 | 1.76 | 0.29 | 37.3 | 111.0 | 232.4 |
| 2_2 (0.7/10000/old) | 1.78 | 26.03 | 27.9 | 29.72 | 10.34 | 3.57 | 0.56 | 0.10 | 30.7 | 83.2 | 171.2 |
| 2_3 (0.7/12000/old) | 1.47 | 16.12 | 40.81 | 27.42 | 9.76 | 2.71 | 1.01 | 0.70 | 37.8 | 83.2 | 170.3 |
| 3_1 (0.8/8000/old) | 3.80 | 21.24 | 21.51 | 33.89 | 12.37 | 5.54 | 1.44 | 0.21 | 30.0 | 94.8 | 196.0 |
| 3_2 (0.8/10000/old) | 5.70 | 20.76 | 22.69 | 32.56 | 13.11 | 3.81 | 1.12 | 0.26 | 27.6 | 91.6 | 185.1 |
| 4_1 (1.0/8000/old) | 3.44 | 11.83 | 23.02 | 34.42 | 16.96 | 8.28 | 1.69 | 0.37 | 38.8 | 105.7 | 217.9 |
| 4_2 (1.0/10000/old) | 12.43 | 13.73 | 30.26 | 29.45 | 9.32 | 3.44 | 0.94 | 0.44 | 12.2 | 82.9 | 171.3 |
| 4_3 (1.0/12000/old) | 15.98 | 22.46 | 30.44 | 22.06 | 5.90 | 1.96 | 0.85 | 0.35 | 7.0 | 70.1 | 140.2 |
| 5_1 (0.6/8000/new) | 7.62 | 6.00 | 27.13 | 36.8 | 14.81 | 6.45 | 0.72 | 0.47 | 33.2 | 101.5 | 201.5 |
| 5_2 (0.6/10000/new) | 11.71 | 10.6 | 30.29 | 30.99 | 10.78 | 4.38 | 0.90 | 0.35 | 14.3 | 87.4 | 182.2 |
| 6_1 (0.7/8000/new) | 6.20 | 10.78 | 16.3 | 33.96 | 18.57 | 11.7 | 2.16 | 0.33 | 31.8 | 113.4 | 243.0 |
| 6_2 (0.7/10000/new) | 3.35 | 10.18 | 23.67 | 35.66 | 15.45 | 9.44 | 1.97 | 0.30 | 42.7 | 106.7 | 228.6 |
| 7_2 (0.8/10000/new) | 8.21 | 11.3 | 32.64 | 31.02 | 10.04 | 5.29 | 1.02 | 0.49 | 26.3 | 88.1 | 188.7 |
| 8_1 (1.0/8000/new) | 6.04 | 10.27 | 19.05 | 32.62 | 16.93 | 11.04 | 3.40 | 0.66 | 32.8 | 111.1 | 251.7 |
| 8_2 (1.0/10000/new) | 7.63 | 10.92 | 25.93 | 32.32 | 13.36 | 7.47 | 1.91 | 0.46 | 27.8 | 97.9 | 214.0 |
| HME (40%) Lot. | 24 | 21 | 32 | 18 | 3 | 1 | 1 | 0 | 3.7 | 64.0 | 125.9 |
| 11122754 | | | | | | | | | | | |

*Dₓ was defined by Eq. 2) and 3).
$\begin{matrix}Z = log {D}_{j - 1} + ( log {D}_{j} - log {D}_{j + 1} ) \times \frac{x - {Q}_{j + 1}}{Qj - {Q}_{j + 1}} \\ ( 2 ) \\ {D}_{x} = {10}^{z} . \\ ( 3 )\end{matrix}$ Dₓ is particle size at arbitrary cumulative %
D is particle size. Dj is larger than Dⱼ₊₁ and Dₓ should be between Dⱼ₊₁ and Dⱼ.
Q is cumulative % of each particle size, D.

TABLE 14
Summary of Milling study

| | | | | | | | | % | | | | | | | |
|---|---|---|---|---|---|---|---|---|---|---|---|---|---|---|---|
| | | Screen | Milling | | HME PSD | | | HME | | | | | | | |
| | Milling | size | speed | Rotor | >250 | 250- | <75 | in | MCC | Tablet hardness [N] | | | | Evaluation @7 kN | |
| | No. | [mm] | [rpm] | Type | um | 75 μm | um | Tab. | grade | 5 kN | 7 kN | 9 kN | 11 kN | Hardness* | Dissolution** |
| 1 | 4_1 | 1 | 8000 | Old | 10.34 | 74.4 | 15.27 | 50 | UF702 | 86 | 132.6 | 190.8 | 216.3 | YES | YES |
| 2 | | | | | | | | 50 | UF711 | 104.9 | 147 | 173.3 | — | YES | YES |
| 3 | | | | | | | | 60 | UF702 | 62.4 | 95.6 | 107.7 | 168.6 | NO | YES |
| 4 | | | | | | | | 60 | UF711 | 74.7 | 108 | 151 | — | YES | YES |
| 5 | 4_2 | 1 | 10000 | Old | 4.81 | 69.03 | 26.16 | 50 | UF702 | 105.8 | 154 | 207.1 | 249.3 | YES | YES |
| 6 | | | | | | | | 50 | UF711 | 111.9 | 163.8 | 219 | — | YES | YES |
| 7 | | | | | | | | 60 | UF702 | 82.7 | 126 | 167.8 | 209.2 | YES | NO |
| 9 | 4_3 | 1 | 12000 | Old | 3.16 | 58.4 | 38.44 | 50 | UF702 | 107.2 | 164.8 | 219.8 | 177.4 | YES | NO |
| 11 | | | | | | | | 60 | UF702 | 89.2 | 141 | 178.7 | 140.2 | YES | NO |
| 13 | 5_1 | 0.6 | 8000 | New | 7.63 | 78.74 | 13.63 | 50 | UF702 | 82 | 126.2 | 174.9 | 213.8 | YES | YES |
| 14 | | | | | | | | 50 | UF711 | 106.5 | 156.7 | 210.2 | — | YES | YES |
| 15 | | | | | | | | 60 | UF702 | 69.8 | 109.8 | 149.4 | 116.9 | YES | NO |
| 17 | 5_2 | 0.6 | 10000 | New | 5.64 | 72.05 | 22.31 | 50 | UF702 | 86.9 | 132.3 | 181.5 | 234 | YES | YES |
| 18 | | | | | | | | 50 | UF711 | 110.3 | 168 | 218.1 | — | YES | YES |
| 19 | | | | | | | | 60 | UF702 | 68.9 | 108.5 | 148.5 | 190.3 | YES | NO |
| 21 | 6_1 | 0.7 | 8000 | New | 14.19 | 68.83 | 16.98 | 50 | UF702 | 76.4 | 115.3 | 161.7 | 199 | YES | YES |
| 22 | | | | | | | | 50 | UF711 | 96 | 144.8 | 180.3 | 148.3 | YES | YES |
| 23 | | | | | | | | 60 | UF702 | 51.8 | 85.7 | 116.5 | — | NO | YES |
| 24 | | | | | | | | 60 | UF711 | 69 | 107.4 | 140 | — | YES | YES |
| 25 | 6_2 | 0.7 | 10000 | New | 11.7 | 74.77 | 13.52 | 50 | UF702 | 86.8 | 139.7 | 183.7 | 221.6 | YES | YES |
| 27 | | | | | | | | 60 | UF702 | 73.3 | 112.1 | 176.6 | 155.8 | YES | NO |
| 33 | Conv_1 | 0.5 | 10000 | Old | 2 | 53 | 45 | 50 | UF702 | 113.8 | 170 | 234 | 266.9 | YES | NO |
| 35 | | | | | | | | 60 | UF702 | 93.9 | 143.5 | 194.5 | 239.6 | YES | NO |

*Tablet hardness ≥100 N = ‘YES’,
**Dissolution ≥75% released in 45 min = ‘YES’

### Example 6

### Scale-Up of HME (50%)

A scale-up study with the HME (50%) formulation was performed. In this study, HME process development was conducted to find the optimal manufacturing process parameters and check the scale-up feasibility with large scale Extruder (Leistritz 18 mm) for GMP manufacturing. A long run HME process with optimized HME process conditions at 10 kg scale was also conducted.

### Sample Preparation

Compound 1 and copovidone were mixed with High shear mixer. The powder mixture was fed into the Hot Melt Extruder (Leistritz 18 mm) with various process conditions to optimize the process conditions. And resulting extrudate strand was cooled by a cooling belt with air flow. The extrudate strand from process optimization was manually milled by mortar and pestle for the analysis, and the strand from long run process was milled by Pin-mill with the following milling conditions: Screen size: 0.7 mm, Milling speed: 8000 rpm, Blade type: blade rotor. During HME manufacturing, extrudate outlet flow behavior from Die and visual appearance of the strands were checked on site. Representative HME process conditions of this study are shown in Table 15.

### Results

The results summary of the scale-up study with HME (50%) is presented in Table 16. There was no degradation in all the samples. The impact of each HME process conditions on the manufacturability and quality was summarized as follows:

Feeding rate: Faster gave better productability, but sometimes white dots were observed because of shorter residence time. Collected samples were very hot because of the insufficient cooling system capability.

Screw speed: Higher was lower white dots risk, but the strand color became more brownish because of the higher mechanical shear.

Temperature: Higher was lower white dots risk, but the enantiomer and strand color became higher and more brownish. At least 180° C. was necessary to achieve good Extrudate outlet flow from die exit because HME (50%) was sticker than HME (40%).

The selected HME process conditions for long run are (102) in Table 15.

Chemical and physical properties of 40% and 50% HME powder which was manufactured in the long run and final demo batch, respectively, are shown in Table 17. There was no API peak in both mDSC and XRPD (X-ray Powder Diffraction), and the HME showed comparable and equivalent trend with HME (40%). The repeatability of the milling process with the new milling conditions which were set in the milling study (was confirmed, and the HME PSD met the target, “% of <75 μm HME particle: <30%”. The summary of stability study and solid state characterization are followings;

HME (50%) powder was stable (no degradation and re-crystallization) and similar trend to HME (40%) in any storage conditions including photo stability.

The solid-state properties (XRPD, SEM, PSD, FT Raman and DSC) of HME (50%) were equivalent to HME (40%) (20140399), and the part of data is shown in Table 17. HME (40%) (20140399) represents a sample milled using the conditions described in WO 2015/148828.

TABLE 16
Results summary for scale-up study of HME (50%)

| | Assay | LOD* | Impurity | Enantiomer | | |
|---|---|---|---|---|---|---|
| No. | [%] | [%] | [%] | [%] | XRPD | Observation |
| 2-1 | 94.1 | 3.72 | <0.05 | 0.89 | no peak | White dots |
| | | | | | | Bad flow |
| 2-2 | 93.0 | | <0.05 | 0.75 | | White dots |
| | | | | | | Bad flow |
| 2-3 | 94.6 | | <0.05 | 0.70 | | Good |
| 2-4 | 94.7 | | <0.05 | 0.71 | no peak | Good |
| 2-5 | 95.0 | | <0.05 | 0.70 | | White dots |
| 2-6 | 94.7 | | <0.05 | 0.89 | | Good |
| 2-7 | 95.4 | | <0.05 | 0.93 | | Brownish |
| 2-8 | 95.1 | | <0.05 | 0.61 | no peak | White dots |
| | | | | | | Bad flow |
| 2-9 | 94.5 | | <0.05 | 0.72 | | Good |
| 2-10 | 94.3 | | <0.05 | 0.51 | | Good |
| 2-11 | 94.0 | | <0.05 | 0.55 | | Good |
| 2-12 | 94.3 | | <0.05 | 0.60 | | Good |
| 2-13 | 95.6 | | <0.05 | 0.51 | | Insufficient |
| | | | | | | cooling |
| 2-14 | 94.3 | 2.95 | <0.05 | 0.46 | no peak | Insufficient |
| | | | | | | cooling |

*Loss on Drying

TABLE 17
Chemical and physical properties of long run for HME (50%) (No. 10_2)

| | | HME (40%) | HME (50%) |
|---|---|---|---|
| Test items | | (20140399) | (10_2) |
| Assay [%] | | 96.4 | 99.0 |
| Impurity [%] | | <0.05 | <0.05 |
| Enantiomer [%]* | | 0.9 | 0.59 |
| Enantiomer trend | 0 | N/A | 0.7 |
| [min, %] | 5 | | 0.67 |
| | 10 | | 0.67 |
| | 20 | | 0.46 |
| | 30 | | 0.54 |
| | 60 | | 0.53 |
| | 120 | | 0.55 |
| | 180 | | 0.62 |
| | 240 | | 0.58 |
| | 300 | | 0.65 |
| | 360 | | 0.71 |
| mDSC or DSC, | | 105 | 106.527 |
| Tg (onset) [° C.] | | (DSC) | (mDSC) |
| XRPD | | no peak | no peak |
| PSD [%] | >500 | 1 | 0 |
| | 500-355 | 1 | 2 |
| | 355-250 | 2 | 9 |
| | 250-180 | 5 | 18 |
| | 180-106 | 25 | 30 |
| | 106-75 | 22 | 27 |
| | 75-45 | 25 | 8 |
| | <45 | 19 | 6 |
| PSD [μm] | D₁₀ | 5.1 | 36.7 |
| | D₅₀ | 67.5 | 103.8 |
| | D₉₀ | 140.4 | 223.3 |

*Pre-Extrusion: 0.17%

### Example 7

### Manufacturing of Prototype Tablets (20, 70, 100 and 150 mg) with HME (50%)

By using HME (50%) which was manufactured at the long run process, prototype HME (50%) core tablets (20, 70, 100 and 150 mg) were manufactured at lab scale in FD. Tablet properties and dissolution of tablets with various compression forces were evaluated to set the target tablet hardness and thickness.

### Sample Preparation

HME (50%) was blended with MCC, croscarmellose sodium, colloidal silicon dioxide with mortar and pestle. And then the blended powder was put into a glass bottle with magnesium stearate and shake it gently for 100 times at 20 tabs scale. The blended powder was compressed on a single hand tablet press (HANDTAB-200, Ichihashi seiki) into tablets with various compression forces. The tablet properties and dissolution were evaluated. Prototype HME (50%) core tablet formulation (20, 70, 100 and 150 mg) are shown in Table 18.

TABLE 18
Prototype HME (50%) core tablet formulation (20, 70, 100 and 150 mg)

| Dose strength | 20 mg | | 70 mg | | 100 mg | | 150 mg | |
|---|---|---|---|---|---|---|---|---|
| Tablet weight | 160 mg | | 350 mg | | 500 mg | | 625 mg | |
| Tablet shape | Round | | Caplet | | Oval (Caplet)* | | Oval | |
| | 7.5 mm φ | | 13 × 7 mm | | 14 × 8 mm | | 16 × 9 mm | |
| | | | | | (14 × 9 mm)* | | | |
| Lot No. | 20160118-1 | | 20160118-2 | | 20151218-3 | | 20151218-4 | |
| | | | | | (20151218-5) | | | |
| | % (w/w) | mg/tab | % (w/w) | mg/tab | % (w/w) | mg/tab | % (w/w) | mg/tab |
| HME (50%) | 25 | 40 | 40 | 140 | 40 | 200 | 48 | 300 |
| Compound 1 | 12.5 | 20 | 20 | 70 | 20 | 100 | 24 | 150 |
| VA64 | 12.5 | 20 | 20 | 70 | 20 | 100 | 24 | 150 |
| Microcrystalline Cellulose | 69 | 110.4 | 51 | 178.5 | 51 | 255 | 43 | 268.75 |
| (UF711) | | | | | | | | |
| Croscarmellose Sodium | 5 | 8 | 8 | 28 | 8 | 40 | 8 | 50 |
| Colloidal Silicon Dioxide | 0.5 | 0.8 | 0.5 | 1.75 | 0.5 | 2.5 | 0.5 | 3.125 |
| Magnesium Stearate | 0.5 | 0.8 | 0.5 | 1.75 | 0.5 | 2.5 | 0.5 | 3.125 |
| Total | 100 | 160 | 100 | 350 | 100 | 500 | 100 | 625 |

*Two different shapes of table were prepared for 100 mg core tablet

### Results

The tablet properties and dissolution profiles are shown in Table 19 and FIG. 7. As a compression force increased the tablet hardness increased and reached the target hardness range, 150-200 N. The risk of capping/sticking during compression seemed to be low in this compression force range because a linearity between compression force and hardness was confirmed. Furthermore, all HME (50%) core tablets showed quick dissolution profile regardless of compression force because disintegration of HME (50%) core tablets was improved by HME PSD optimization. A decrease of dissolution speed by larger HME particles was not observed. All these prototype HME (50%) core tablets were manufacturable in the dose range, 20-150 mg.

TABLE 19
Tablet properties of prototype HME (50%) core tablets (20, 70, 100 and 150 mg)

| | | | Com- | | | Diameter | |
|---|---|---|---|---|---|---|---|
| | Tablet size | | pression | | Thick- | (Major | Hard- |
| Dose | (dimension, | | force | Weight | ness | axis) | ness |
| strength | shape) | | kN | mg | mm | mm | N |
| 20 mg | 7.5 | Round | 4 | 165.6 | 4.17 | 7.55 | 95.4 |
| | mmφ | | 5 | 163.9 | 4.00 | 7.56 | 142.5 |
| | | | 6 | 166.2 | 3.94 | 7.56 | 169.3 |
| | | | 7 | 162.1 | 3.81 | 7.55 | 183.9 |
| 70 mg | 13 × | Caplet | 7 | 353.8 | 5.11 | 13.12 | 177.3 |
| | 7 mm | | 9 | 354.6 | 4.94 | 13.11 | 218.5 |
| 100 mg | 14 × | Oval | 7 | 502.2 | 6.16 | 14.10 | 235.1 |
| | 8 mm | | 9 | 504.2 | 5.95 | 14.12 | 278.8 |
| | 14 × | Caplet | 5 | 503.0 | 5.93 | 14.16 | 116.5 |
| | 9 mm | | 7 | 505.4 | 5.60 | 14.16 | 168.8 |
| | | | 9 | 505.5 | 5.41 | 14.14 | 210.9 |
| 150 mg | 16 × | Oval | 5 | 630.7 | 6.76 | 16.17 | 105.6 |
| | 9 mm | | 7 | 636.3 | 6.42 | 16.18 | 143.5 |
| | | | 9 | 630.7 | 6.13 | 16.16 | 197.6 |

TABLE 20
HME (50%) tablet formulation for scale-up study (100 and 150 mg)

| Dose strength | 100 mg | 150 mg | | | | | |
|---|---|---|---|---|---|---|---|
| Core tablet weight | 500 mg | 625 mg | | | | | |
| Tablet shape | 14 × 8 mm, Oval | 16 × 9 mm, Oval | | | | | |
| Lot No. (Core tablets) | 20160328-1 | 20160116 | | | | | |
| Lot No. (FC tablets) | 20160328-11 | 20160328-21 | | | | | |
| | 20160328-12 | 20160328-22 | | | | | |
| | | | | Theoretical | | | Theoretical |
| | | | | Weights | | | Weights |
| | | % (w/w) | mg/tab | (g) | % (w/w) | mg/tab | (g) |

| | | | | | | |
|---|---|---|---|---|---|---|
| HME (50%) | 40 | 200 | 400 | 48 | 300 | 2400 |
| API | 20 | 100 | 200 | 24 | 150 | 1200 |
| VA64 | 20 | 100 | 200 | 24 | 150 | 1200 |
| MCC (UF711) | 51 | 255 | 510 | 43 | 268.75 | 2150 |
| Croscarmellose Sodium | 8 | 40 | 80 | 8 | 50 | 400 |
| Colloidal Silicon Dioxide | 0.5 | 2.5 | 5 | 0.5 | 3.125 | 25 |
| Magnesium Stearate | 0.5 | 2.5 | 5 | 0.5 | 3.125 | 25 |
| Sub-total | 100 | 500 | 1000 | 100 | 625 | 5000 |
| Opadry White | 3.2 | 16 | 32 | | | |
| 03F480011 | | | | | | |
| Opadry Yellow | | | | 2.2 | 14 | 112 |
| 03F42240 | | | | | | |
| Opadry Red 03F45081 | | | | 1.1 | 7 | 56 |
| Total | | 516 | 1032 | | 646 | 5168 |

### Example 8

### Scale-Up of HME (50%) Tablet Formulation

Scale-up of prototype HME (50%) tablets (100 and 150 mg) was conducted with HME (50%) which was manufactured in a scale-up long run process.

### Sample Preparation

For the 100 mg tablet, HME (50%) was blended with MCC, croscarmellose sodium, colloidal silicon dioxide and magnesium stearate in a bag at FD. And then the blended powder was compressed on rotary tablet press into tablets with various compression forces at 1 kg scale.

For 150 mg tablet, HME (50%) was blended with MCC, croscarmellose sodium, colloidal silicon dioxide and magnesium stearate in a blender. The blended powder was compressed on rotary tablet press into tablets with various compression forces at 5 kg.

Film coating of both core tablets was conducted by using a film coater with different colors to have distinguishability by color contrast between these dose strengths. 150 mg tablet color was orange (mixture of Opadry red and yellow) and 100 mg was white (Opadry white). A stability study and solid state analysis were conducted to confirm comparability between JME (40%) and HME (50%) film coated tablets. The BU (Blending Uniformity), CU (Content Uniformity), tablet properties and dissolution profile were also evaluated to find the appropriate manufacturing process parameter range. HME (50%) tablet formulation (100 and 150 mg) for scale-up study is shown in Table 20.

### Results

The BU and CU results are shown in Table 21.

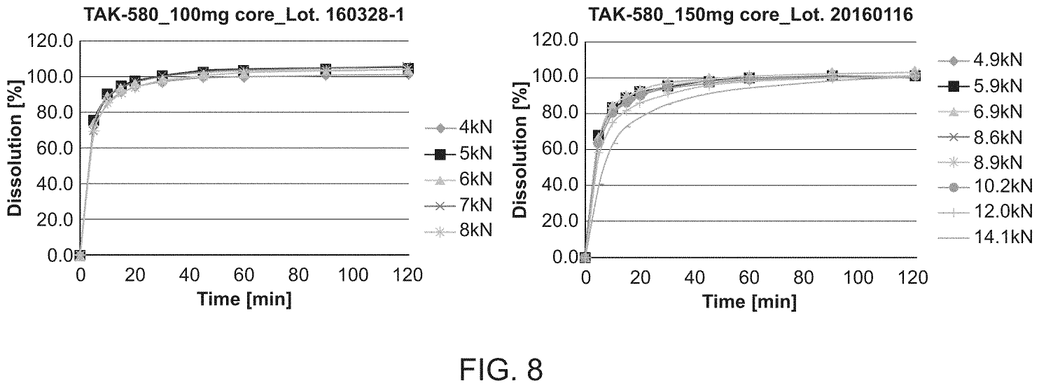

Tablet properties and dissolution profiles of the HME (50%) tablets (100 and 150 mg) compressed with different compression force are shown in Table 22, Table 23, and FIG. 8. From these results, HME (50%) tablets (100 and 150 mg) manufactured with a rotary tablet press showed similar tablet properties and dissolution profiles to prototype HME (50%) tablets manufactured at lab scale, and met the desired quality target and acceptance criteria. Tablets with lower compression force had chippings on the core tablet surface and edge after friability testing. Tablets with higher compression force showed slower dissolution profile and disintegration due to the hydro-gel matrix formation in dissolution media. Therefore, 6.0 and 8.9 IN were set as the target compression force of 100 and 150 mg core tablets respectively.

TABLE 21
Blending uniformity (BU) and content uniformity
(CU) of HME (50%) tablets (100 and 150 mg)

| | 100 mg | | 150 mg | |
|---|---|---|---|---|
| n = 10 | BU* | CU | BU | CU |
| AVE | — | 100.50% | 98.20% | 99.00% |
| Min | — | 97.40% | 99.80% | 101.10% |
| Max | — | 104.10% | 97.20% | 97.8% |
| SD | — | 1.58% | 0.80% | 1.00% |

*Blended in PE bag

TABLE 22
Tablet properties of HME (50%) core tablets (100 mg)

| | 100 mg core tablets | | | | |
|---|---|---|---|---|---|
| Compression | | | 6 kN | | |
| force | 4 kN | 5 kN | (Center) | 7 kN | 8 kN |
| Hardness | 152.33N | 184.53N | 227.65N | 240.90N | 288.93N |
| Thickness | 6.67 mm | 6.45 mm | 6.28 mm | 6.16 mm | 6.04 mm |
| Friability | 0.02 %** | 0.00% | 0.01% | 0.00% | 0.00% |

**chipping

TABLE 23
Tablet properties of HME (50%) core tablets (150 mg)

| | 150 mg core tablets | | | | | | | |
|---|---|---|---|---|---|---|---|---|
| Compression | | | | | 8.9 kN | | | |
| force | 4.9 kN | 5.9 kN | 6.9 kN | 8.6 kN | (Center) | 10.2 kN | 12.0 kN | 14.1 kN |
| Hardness | 86 N | 104 N | 121 N | 150 N | 174 N | 199 N | 233 N | 270 N |
| Thickness | 6.56 mm | 6.40 mm | 6.29 mm | 6.09 mm | 6.03 mm | 5.87 | 5.77 mm | 5.67 mm |
| Friability | 0.13%** | 0.04%** | 0.02%** | 0.01% | 0.00% | 0.00% | 0.00% | 0.01% |

**chipping

It is to be understood that the foregoing embodiments and exemplifications are not intended to be limiting in any respect to the scope of the disclosure, and that the claims presented herein are intended to encompass all embodiments and exemplifications whether or not explicitly presented herein.

All patents and publications cited herein are fully incorporated by reference in their entirety.

1.-49. (canceled)

50. A pharmaceutical composition comprising:
(1) a solid dispersion having a D₅₀ of about 75 μm to about 400 μm; and
(2) one or more pharmaceutically acceptable excipients, wherein the solid dispersion comprises:
(a) about 10% w/w to about 70% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof; and
(b) about 30% w/w to about 90% w/w of a polymer.

51. The pharmaceutical composition of claim 50, wherein the polymer is vinylpyrrolidone-vinyl acetate copolymer.

52. The pharmaceutical composition of claim 51, wherein the vinylpyrrolidone-vinyl acetate copolymer is copovidone.

53. The pharmaceutical composition of claim 50, wherein the polymer is polyvinylpyrrolidone, vinylpyrrolidone-vinyl acetate copolymer, cross linked polyvinyl N-pyrrolidone, polyvinyl alcohol (PVA), polysaccharide, hydroxypropyl methylcellulose, hydroxyethyl cellulose (HEC), hydroxypropyl cellulose (HPC), polyethylene oxides (PEOs), hydroxypropyl-β-cyclodextrin (HP-β-CD), sulfobutylether-β-cyclodextrin, hydroxypropyl methylcellulose acetate succinate (HPMC-AS-HF), polyethylene glycol (PEG), polyvinyl caprolactam-polyvinyl acetate-polyethylene glycol graft copolymer (PVAc-PVCap-PEG), poly(lactide-co-glycolide) (PLGA), cellulose-esters, cellulose-acrylates, poly (ethylene-co-vinyl acetate), poly-methacrylate derivatives, poloxamers, polylactic acid (PLA), poly(glycolide) (PGA), or any combination thereof.

54. The pharmaceutical composition of claim 50, wherein the solid dispersion has a D₅₀ of about 85 μm to about 250 μm.

55. The pharmaceutical composition of claim 50, wherein the solid dispersion has a D₅₀ of about 95 μm to about 150 μm.

56. The pharmaceutical composition of claim 50, wherein the solid dispersion comprises about w/w to about 65 w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof.

57. The pharmaceutical composition of claim 50, wherein the one or more pharmaceutically acceptable excipients comprise a filler, a disintegrant, a glidant, a lubricant, or a combination thereof.

58. The pharmaceutical composition of claim 50, comprising about 40% w/w to about 90% w/w of one or more pharmaceutically acceptable excipients.

59. The pharmaceutical composition of claim 50, wherein the (R)-2-(1-(6-amino-5-chloropyrimidine-4-carboxamido)ethyl)-N-(5-chloro-4-(trifluoromethyl)pyridin-2-yl)thiazole-5-carboxamide is amorphous.

60. A process for preparing the pharmaceutical composition, the process comprising:
(1) admixing (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof, and a vinylpyrrolidone-vinyl acetate copolymer to give a powder mixture;
(2) subjecting the powder mixture to hot melt extrusion to give a solid dispersion extrudate;
(3) milling the solid dispersion extrudate to give a solid dispersion having a D₅₀ of about 75 μm to about 400 μm; and
(4) admixing the solid dispersion with one or more pharmaceutically acceptable excipients.

61. The process of claim 60, wherein the vinylpyrrolidone-vinyl acetate copolymer is copovidone.

62. The process of claim 60, wherein the solid dispersion has a D₅₀ of about 85 μm to about 250 μm.

63. The process of claim 60, wherein the solid dispersion has a D₅₀ of about 95 μm to about 150 μm.

64. The process of claim 60, wherein the solid dispersion comprises about 35 w/w to about 65% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof.

65. The pharmaceutical composition of claim 60, wherein the one or more pharmaceutically acceptable excipients comprise a filler, a disintegrant, a glidant, a lubricant, or a combination thereof.

66. A method of treating a patient in need thereof, the method comprising administering to the patient a therapeutically effective amount of the pharmaceutical composition comprising:
(1) a solid dispersion having a D₅₀ of about 75 μm to about 400 μm; and
(2) one or more pharmaceutically acceptable excipients, wherein the solid dispersion comprises:
(a) about 10% w/w to about 70% w/w of (R)-2-(1-(6-amino-5-chloropyrimidine-4-carboxamido)ethyl)-N-(5-chloro-4-(trifluoromethyl)pyridin-2-yl)thiazole-5-carboxamide, or a pharmaceutically acceptable salt thereof; and
(b) about 30% w/w to about 90% w/w of a polymer;
wherein the patient has cancer.

67. The method of claim 66, wherein the cancer has a BRAF gene mutation, a NRAS gene mutation, or a BRAF gene mutation and a NRAS gene mutation.

68. The method of claim 66, wherein the cancer has a V600 BRAF gene mutation.

69. The method of claim 66, wherein the cancer is selected from the group consisting of skin cancer, ocular cancer, gastrointestinal cancer, thyroid cancer, breast cancer, ovarian cancer, lung cancer, brain cancer, laryngeal cancer, cervical cancer, lymphatic cancer, genitourinary cancer, and bone cancer.
